(19) United States
(12) Patent Application Publication
(10) Pub. No.: US 2006/0025713 A1
(43) Pub. Date: Feb. 2, 2006

(54) Magnetic particle-based therapy

(76) Inventors: Alex Rosengart, Chicago, IL (US); Michael D. Kaminski, Lockport, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/844,180

(22) Filed: May 12, 2004

Related U.S. Application Data
(60) Provisional application No. 60/469,765, filed on May 12, 2003.

(51) Int. Cl. A61M 37/00; A61K 49/00

(52) U.S. Cl. 604/5.02; 604/5.04; 424/9.1

(57) ABSTRACT

The invention provides materials and methods for the administration of an effectively magnetic medication or diagnostic reagent, or for the removal, sequestration, or effective conversion to a non-deleterious condition of a deleterious substance such as a toxin (e.g., biological, chemical, or radiological compound or composition) in vivo by administering a biocompatible magnetic particle to an organism in need, e.g., by delivery to the bloodstream, with the organism optionally having an internal magnetizable stent or magnetizable seed. The materials and methods are useful in the diagnosis and treatment of a variety of acute and chronic diseases, disorders and conditions afflicting man and other organisms, as well as for the removal of a variety of deleterious substances, including toxins, with the optional aid of an external magnetic generator and an optional magnetic filtration device.

This application claims the benefit of U.S. Provisional Patent Application No. 60/469,765, filed May 12, 2003.

The U.S. Government has a paid-up license in this invention and the right in limited circumstances to require the patent owner to license others on reasonable terms as provided for by the terms of contract no. W-31-109-ENG-38 awarded by the U.S. Department of Energy.

### BACKGROUND OF THE INVENTION

Therapeutic approaches to improve the health and/or well being of organisms frequently involve the controlled introduction of therapeutic compounds to, or the removal of deleterious compounds from, organisms in need of treatment. The wide range of therapeutics currently in use, and the extensive efforts to develop additional therapeutics, attests to the significance of health-related technologies today. The continuing progress in developing therapies to address an increasing number of diseases and disorders in, e.g., mammals such as humans, is tempered by new challenges that constantly arise. One set of challenges involves the administration of therapeutics, or the removal of deleterious compounds, from an organism in need. As our understanding of the particular organs, tissues and cells in need of treatment advances, the need for tailored therapeutic delivery, or deleterious compound removal, becomes increasingly urgent.

One approach to tailored drug delivery is the use of drug-eluting stents, which are increasingly advocated for not only providing endoluminal patency but also for delivering medication within the vicinity of the stent. Stents have proven useful when placed into a variety of lumina within the body, such as the arteries and veins of the vasculature, the lymphatic system, the liver biliary and intestinal tracts, as well as respiratory and genitourinary systems. However, several inherent limitations exist—importantly, uncontrollable drug-release pharmacokinetics and the inability to change the choice and delivery mode of the drug once the stent is in place. Additionally, typical stent materials advocated for medical and veterinary applications are paramagnetic and are, therefore, not magnetizable within magnetic fields. A further variation of stent technology and a less commonly explored form of targeted drug delivery than stent-based approaches is a seed-based approach, which involves premedicated materials implanted into body cavities or tissues in order to treat various diseases, disorders or conditions, such as certain tumors. For example, radioactive seeds are surgically implanted into a non-resectable brain tumor to treat surrounding cancer tissue with irradiation. The implantable seed materials in current use are non-magnetic, are not capable of delivering more than one medication to the surrounding tissue, have a limited pharmacological half-life, and cannot be adjusted to deliver the drug over certain time periods.

On a different note, effective removal of harmful substances from humans provides, in many instances, not only a life-saving procedure as in acute intoxications but also a valuable treatment option to reduce mortality and morbidity from many chronic diseases and exposures. Typical examples elucidating the need for toxin removal (detoxification) from humans includes, but are not limited to, acute intoxications, i.e., from pharmacological substances, biohazardous exposures, and overwhelming infections, as well as chronic disease states such as autoimmune diseases.

Generally, one method of detoxification of humans or animals is clearing the bloodstream of the offending agents. Several methods for such clearing are currently employed and can be conveniently grouped into endogenous and exogenous clearance methods. Endogenous methods facilitate the internal degradation or excretion of toxins, whereas exogenous techniques are commonly based on detoxifying human blood. Unfortunately, most endogenous methods have a low therapeutic value and existing exogenous clearing techniques typically provide only non-specific detoxification and therefore only limited clinical usefulness. In addition, all these methods have the potential of serious side effects further limiting their clinical utility. Unfortunately, many toxins and biohazards currently cannot be removed from exposed humans and therapy is limited to supportive measures. A method for selective and quantitative detoxification is urgently needed.

Current state-of-the-art methods for endogenous and exogenous toxin and biohazard removal from humans can be summarized as follows: (A) Hemodialysis and Hemofiltration, which apply an osmotic gradient across a semi-permeable membrane to dialyze/filter hydrophilic substances out of the blood. The major limitations are long procedure duration, extracorporeal circulation of large blood volumes requiring large-bore arterial access, non-selective substance removal, and effectiveness limited to hydrophilic substances of lower molecular weight. Its use is mostly restricted to patients with kidney failure and in some medication-related intoxications. (B) Plasmapheresis utilizes extracorporeal, non-specific exchange of plasma (i.e., cell-free blood) with albumin or saline solutions. This method removes most of the blood fluid phase and therefore can only be used for a limited period of time and in specific clinical situations where the toxic substance is present in abundant concentration. Its utility is generally restricted to autoimmune diseases. (C) Extracorporeal Immunoabsorption, a variation of hemodialysis in which extracorporeal circulated blood is exposed to a larger exchange surface saturated with immune absorbent materials (e.g., antibodies). It is a more specific removal method but less effective than simple hemodialysis, requires the circulation of large blood volumes, and is restricted to specific antibody-antigen interactions. (D) Direct Injection of Chelators and Antibodies, in which, by example, injected antibodies neutralize some actions of a circulating antigen (e.g., medication or bacterial toxin interactions). However, complete antigen binding often cannot be achieved and also relatively high antibody dosing is required, increasing the risk of allergic (anaphylactic) and systemic (kidney failure, and the like) side effects. Furthermore, the antibody-toxin complex is not removed from the blood and remaining toxin can dissociate, leading to rebound intoxication.

Magnetic particle systems have been described that seek to deliver drugs to areas within the body [Volkonsky, et al, U.S. Pat. Nos. 5,549,915; 5,651,989; 5,705,195; and 5,200,547]. Magnetic particles are also well established in the field of bioassay and biological separations [Cortex Biochem, Inc.]. Moreover, liposomes have been designed for prolonged circulation in the body and functionalization of liposomic [Allen, et al., Adv. Drug Deliv. Rev., 16, 267-284, 1995; Huang et al, Cancer Research, 52, 6774, 1992] and magnetic particle surfaces have been described [Hafeli et al., Journal of Biomedical Materials Research, 28, 901, 1994].

In terms of toxin removal, U.S. Pat. No. 5,123,901 discloses an in vitro technology using magnetic particles to remove toxins from blood. More particularly, the '901 patent discloses the use of magnetic particles composed of polystyrene in an extracorporeal mixer and magnetic separator. In such a system, there are fewer constraints imposed on the composition (material) and configuration (size, surface charge, surface groups) of the particles. The in vitro technology avoids particle size requirements because the particles do not have to pass through the capillary beds or avoid loss in organ fenestrations (filters). Therefore, the technique disclosed in the '901 patent can use particles of any size deemed necessary for functional group attachment and magnetic separation. The in vitro technology avoids addressing the particular mode of functional group attachment (receptors like antibodies or antitoxins) because they may be attached to the surface directly; further, the in vitro technology need not be concerned about immunoreactivity. The apparent advantages of in vitro technologies over in vivo technologies is illusory, however, because of the time and expense required to implement in vitro technologies. Additionally, in vitro technologies are recognized in the art as presenting risks associated with placing the relevant biological material (e.g., blood) in an in vitro environment, which is necessarily an abnormal ex vivo environment. Further, in vitro technologies do not present the promise of versatility that is characteristic of in vivo technologies, insofar as some deleterious substances may not be amenable to transfer from the in vivo environment to the in vitro environment (e.g., equilibrium favoring organ storage over blood presence for some toxins). Thus, a need continues to exist for in vivo technologies for removing deleterious substances from organisms, including particle-based in vivo technologies.

Magnetic particles also have been contemplated for use in vivo. The '901 patent suggests the in vivo use of dextran-coated microparticles. At the time of issuance of the '901 patent, knowledge in the art focused on particle surface charge as the primary characteristic that defined whether the in vivo delivery of particles could survive in the vasculature. Since that time it has become known that biostabilization is much more complex than simply defining a surface charge near neutral or slightly negative (long-chain dextran will provide a zeta-potential of −4 mV at pH 5). Although dextran is biocompatible (non-toxic) and biodegradable (slowly degrades into fragments), it is not biostabilized. This is significant because biostability means that the particles can avoid recognition by the immune system and subsequent loss through macrophage engulfment. If the particles are not biostable, then they will be removed from circulation within minutes (primarily in the liver). Dextran is not a biostable biopolymer. Thus, injection of dextran nanoparticles into the vasculature will result in immediate removal by the immune system, interfering with the ability of such particles to bind to a deleterious substance such as a toxin and making recovery of the particles more difficult, if not impossible. The '901 patent also includes poly(methylmethacrylates) and derivatives as alternatives to dextran. These have been found to be equally ineffective in in vivo studies [Kreuter, et al., J. Pharm. Sci. 1983, 72:1146-1149; see also Table 5, Chapter 14, page 412-413, in Microspheres Microcapsules&Liposomes, Volume 2: Medical and Biotechnology Applications, Arshady, R. Ed., Citus Books, London, United Kingdom, (1999).]

Paramagnetic agents have been used as a contrast medium for in vivo magnetic resonance imaging (MRI), or as drug delivery vehicles, but not as vehicles for the effective removal of deleterious substances such as toxins. See, e.g., U.S. Pat. No. 5,766,572. In MRI, for example, gadodiamide, a paramagnetic agent (diethylenetriamine pentaacetic acid bismethylamide), is used to induce changes in radiofrequency signals during MRI imaging in order to more accurately visualize the vasculature. However, this medication cannot be used in detoxification methods, as gadodiamide is rapidly distributed within the blood (3.7±2.7 minutes), with rapid elimination from the blood via the kidney and into the extracellular space. In other words, gadodiamide rapidly leaves the blood capillaries and further distributes into the extracellular tissue fluid; hence, gadodiamide is also used to detect changes in blood vessels, i.e., in tumors and the like. Consequently, gadodiamide is unsuitable for toxin-binding within the vasculature.

Particles greater than about 70 nm would not be cleared by fenestrations and urinated from the body. Instead, they would remain trapped in the vasculature (primarily concentrated in the liver) and would thus cause artifactual imaging and would not be useful as a contrast agent. Also, particles that are too large will provide too much contrast for MRI. The artifacts of particles in the 100+ nm or micrometer range would establish a spatially large area of contrast, essentially ruining the resolution of the imaging technique. Thus, the most suitable particles for imaging are small enough to be naturally excreted or distributed throughout the body (<70 nm) and to avoid the creation of unacceptable artifacts for the imaging technique. Thus, the '572 patent discloses a particle that is not in the dissolved state (>few nm) and is cleared by urination (<50 nm).

Such a particle as that described in the '572 patent would not be well-suited for use detoxification because the particles are too small to remain in the vasculature (<70 nm), have too small a magnetic moment due to their very small size, have not been biostabilized to survive for useful periods of time in the vasculature, and have not been specifically configured to incorporate a biostabilizing polymeric layer and surface receptors. The '572 patent also discloses the use of such small particles (e.g., 10-50 nm) as drug delivery vehicles.

The first-order distribution of microparticles within the blood is determined almost entirely by their size. It is known that following intravenous (iv) administration of microparticles larger than about 5 μm, the particles are almost entirely trapped in the lungs by arteriolar and capillary blockade (Slack et al., J. Pharm. Sci. 70:660 (1981)). Conversely, nanoparticles and microparticles smaller than about 3 μm are rapidly removed, mainly by the Kupffer cells of the liver and spleen as a result of opsonization (protein adsorption) and engulfment by macrophages of the reticuloendothelial system (RES) (Yoshioka et al., Int. J. Pharm. 8:131 (1981)).

Following intra-arterial injection, small microparticles are cleared by the RES, while larger microparticles are sequestered in the first capillary bed encountered. As the microparticles begin to degrade, matrix products are released from the target circulation and gradually accumulate in the RES. This pattern of particle deposition appears to be independent of the nature of the microparticles, such as chemical composition or hydrophilic character. Arshady et al. have shown similar biodistribution data for microparticle types including poly(styrene-divinylbenzene), polyacrylamide, albumin, gelatin, poly(alkyl cyanoacrylate), and poly(methyl methacrylate) [Arshady et al., Targeted Delivery of Microparticulate Carriers, In: R. Arshady Microspheres Microcapsules and Liposomes (vol. 2) pp. 403-432, London, UK: Citus Books (1999)].

Because of the rapid clearance of small magnetic particles, such as the particles disclosed in the '572 patent, such particles would be poor candidates for use in binding-mediated toxin removal in vivo. Insufficient time would be provided for effective binding of the deleterious substance in many instances, and the rapid loss of the particles from the in vivo environment would require costly, cumbersome and risky replenishments. Additionally, the compositions and size constraints of the particles disclosed in the '572 patent would result in an insufficient magnetic moment.

Moreover, the rapid clearance of such particles in the MRI context would minimize any undesirable immune response targeting such particles, but the repeated presentation of such particles to effect deleterious substance removal would require consideration of the immunoreactivity of the particles.

In view of the knowledge in the art, it is perhaps unsurprising that no systems or methods have been described that would be useful for the in vivo binding of toxins by relatively long-circulating magnetic particles (MPs), with the subsequent removal of toxin-bound MPs from, e.g., the blood. More generally, and with the limitations of the existing technologies in mind, it is noted that there is currently no adequate detoxification system and, for the majority of biohazard exposures, for instance, no therapies available other than supportive measures.

Also apparent is the absence of methods and systems for the controlled delivery of therapeutics and/or diagnostics to a cell, tissue, organ, or organ system. Moreover, there are no systems or methods for repeated dosing of a targeted therapeutic or diagnostic agent in the absence of repeated invasive procedures.

Based on the foregoing observations, it is apparent that a need continues to exist in the art for methods of delivering therapeutics to localized target sites and to repeatedly deliver therapeutics over time without the need for invasive procedures attending each administration. It is apparent that a need also continues to exist in the art for removing deleterious biological substances (e.g., toxins) from the blood, which is preferably applicable in acute and chronic intoxications, capable of application in treating a wide variety of exposure scenarios, and suitable for detoxifying biological fluids, e.g., human blood, selectively and quantitatively. In addition, a need continues to exist for versatile detoxification methods capable of physical removal of deleterious substances, e.g., radionuclides, that cannot be effectively sequestered or rendered harmless in vivo, while providing sequestration or effective inactivation for those deleterious substances amenable to such treatment. Further, a need continues to exist for removal methods that have minimal side effects and exhibit improved safety profiles that are minimally cumbersome and portable to accommodate different exposure scenarios such as in-the-field applications, and that are amenable to mass production.

### SUMMARY OF THE INVENTION

The invention satisfies at least one of the aforementioned needs in the art by providing a feasible technique to improve on current shortcomings of present state-of-the-art therapeutic delivery systems or detoxifications. These methods are useful in delivering a therapeutic to a target site in an organism, to targeting therapeutics to one or more sites within an organism, or to facilitating iterative dosing schedules without requiring an invasive procedure for each administration. The methods are also useful in removing, sequestering, or otherwise rendering non-deleterious, a variety of biological substances found in an organism, such as in the blood of a mammal (e.g., human). The methods of the invention generally involve systemic administration (e.g., intravenous injection) of magnetic particles, functionalized with a biostabilizing coating and, preferably, with a specific binding partner, into an organism (e.g., by injection into the bloodstream). Following administration, the magnetic particles collectively bind at least one toxin, recognizing that each particle need not bind a deleterious substance. The removal of the magnetic particles will also effect removal of any bound deleterious substance, such as a toxin, from the organism. Permanent removal is facilitated by an extracorporeal magnetic filter, allowing re-introduction of any biological material (e.g., blood) obtained from the organism during the course of removing the magnetic particles.

In one aspect, the invention provides a method for controlling the administration, of an effectively magnetized compound selected from the group consisting of a medication; a diagnostic agent, a specific binding partner for a deleterious substance and an inhibitor of a deleterious substance comprising: (a) introducing a magnetizable device selected from the group consisting of a magnetizable stent and a magnetizable seed into an organism in need, wherein the device is magnetically associated with the compound; and (b) establishing a magnetic field across the device, thereby capturing the compound. In preferred embodiments, the compound is effectively magnetized by attachment to a biocompatible magnetic particle comprising a mean diameter between 100 and 5,000 nanometers and exhibiting an in vivo half-life of at least fifteen minutes. Typically, the attachment involves at least one covalent bond. The device may be permanently magnetic, thereby providing its own magnetic field, or it may be inducibly magnetic, with an external magnetic field generator establishing a magnetic field across the device. The device is introduced into an organism in need by any method known in the art, including surgical implantation, whether involving conventional surgery or a laparoscopic technique, catheter-mediated implantation, cannula-mediated implantation and stereotactic placement.

Compounds contemplated for use in the methods of the invention drawn to the targeted delivery of medications or diagnostic agents include, but are not limited to, chemotherapeutics, radioactive isotopes, fibrinolytic agents (clot busters), anti-platelet aggregation drugs, as well as gene, viral, or cell therapeutics, as well as various diagnostics such as tumor- or cell-binding proteins and markers. The invention also comprehends systems and methods involving a combination of both diagnostics and therapeutics. For the purpose of detoxification, compounds contemplated as useful to remove deleterious substances include chelators for chemical and radioactive substance removal, receptors (antibodies) for binding of cells, cell products, proteins or other biological hazards within and outside the bloodstream, detoxifying agent(s) exhibiting simple physicochemical attraction to a toxin, and any other known compound that specifically binds to, inactivates, or inhibits a deleterious substance such as a toxin.

Magnetic particles for use in the methods comprise a magnetizable material, such as one or more of the following: magnetite (Fe₃O₄), maghemite (g-Fe₂O₃), metallic iron, cobalt, nickel, permalloy, cobalt ferrite (CoFe₂O₄), NdFeB, SmFe₂, TbFe₂, TbDyFe, NdCo₅, SmCo₅, LaCo₅, CeCo₅ and PrCo₅. Further, the magnetic particle may be coated with a biocompatible polymer selected from the group consisting of poly (lactic co-glycolic acid), poly (lactic acid), a linear polyethylene glycol, a branched polyethylene glycol, a propylene glycol, dextran and albumin, the latter two being a carbohydrate polymer and an amino acid polymer, respectively.

In another aspect, the invention provides a method for controlling the administration of an effectively magnetized compound selected from the group consisting of a medication, a diagnostic agent, a specific binding partner for a deleterious substance and an inhibitor of a deleterious substance comprising: (a) introducing a magnetizable device selected from the group consisting of a magnetizable stent and a magnetizable seed into an organism in need, wherein the device is associated with the compound; (b) administering a therapeutically effective amount of an effectively magnetizable compound to the organism; and (c) establishing a magnetic field across the device, thereby capturing the compound. Preferably, the compound is effectively magnetized by attachment to a biocompatible magnetic particle comprising a mean diameter between 100 and 5,000 nanometers and exhibiting an in vivo half-life of at least fifteen minutes. Suitable forms of administration of the compound include, but are not limited to, intraarterial injection, intravenous injection, intramuscular injection, intraperitoneal injection, subcutaneous injection, transdermal delivery, inhalation, intraluminal spraying and topical administration.

In some embodiments of the above-described methods for controlling the administration of an effectively magnetizable compound, the invention comprehends compounds that are either a specific binding partner for a deleterious substance or an inhibitor of a deleterious substance, and the method further comprises removal of the compound attached to the magnetic particle after a time period sufficient for the compound to bind to the deleterious substance, thereby removing the deleterious substance. The time period sufficient for the compound to bind the deleterious substance will vary depending on the nature of the deleterious substance, the nature of the compound, the type of organism and its health, the concentration of compound and the concentration of toxin in the organism, and other variables known to those of skill in the art and determinable using routine procedures.

Another aspect of the invention provides uses of the above-described magnetic particles attached to compounds, such as medications, in the preparation of a medicament for the treatment of a disease, disorder or condition in an organism in need. An analogous aspect of the invention provides uses of such magnetic particles attached to compounds, such as diagnostic agents, in the preparation of a medicament for the diagnosis of a disease, disorder or condition in an organism in need.

In yet another aspect, the invention provides a kit, or article of manufacture, comprising a compound attached to a magnetic particle as described above and a set of instructions for administration of the compound to treat or diagnose a disease in an organism in need.

Turning to the aspect of the invention drawn to the removal of a deleterious substance, such as a toxin, and in agreement with the clinically most successful acute and chronic detoxification system, hemodialysis, the methods of the invention provide a versatile detoxification system that removes the offending agent(s) from, e.g., the bloodstream. Simple blood biohazard sequestration within the bloodstream, as achieved by toxin-ligand or antibody-antigen binding, is frequently insufficient to protect organisms such as humans from harmful exposure to, e.g., toxin(s). This is exemplified (a) in treatment approaches based solely on in vivo antibody-antigen binding, in which safe antibody treatment is problematic due to 1) reduced antibody affinity, 2) systemic side effects (e.g., antibody-antigen complex-mediated diseases, renal failure, and the like), 3) anti-antibody production, and 4) inherent limitations if higher or repeated antibody injections are required; (b) as well as in cases of radioactive and chemical toxin exposures where ligand-toxin binding does not alter the toxic activity and natural disease courses induced by the deleterious biological substance.

Consistent with the preceding discussion and expected to provide at least one of the enumerated advantages, the aspects of the invention drawn to methods for removing a substance from an organism may comprise: (a) administering a biocompatible magnetic particle to an organism under conditions wherein the particle binds to the substance, and wherein the particle has an in vivo half-life of at least fifteen minutes, and preferably at least thirty minutes; and (b) removing the particle from the organism by exposing the particle to a magnetic field, thereby removing the substance. In some embodiments, the particle specifically binds the substance through a specific binding partner attached to the particle. In these embodiments, the particle has a diameter between about 100 to 5,000 nanometers and exhibits an in vivo half life of at least 15 minutes. Substances suitable for manipulations in accordance with this aspect of the invention include a bacterial cell, a virus, a DNA, a RNA, a prion, a radionuclide, a radioactive material, a metal, a toxin, a protein, a genetic structure (i.e., a unit of heredity composed of a nucleic acid), a toxic metabolite generated by an infectious agent or by an organism's host response, a chemical, a pollutant, a medication, and a cell and/or gene modifying agent. Preferred substances include, but are not limited to, a bacterial cell, a virus, a DNA, a RNA, a prion, a radionuclide, a radioactive material and a metal. In some preferred embodiments, the substance is a toxin, such as a toxin selected from the group consisting of Abrin, Adenylate cyclase, Aerolysin, Aflatoxin, Alpha toxin, Adroctonin, Anthrax toxin, Botulinum toxin (A), Botulinum toxin (B), Botulinum toxin (C), Botulinum toxin (D), Botulinum toxin (E), Botulinum toxin (F), Botulinum toxin (G), C2 toxin, C3 toxin, Cholera enterotoxin CLDT, CFN, Conotoxin-alpha, Conotoxin-alpha-A, Conotoxin-psi, Conotoxin-omega, Conotoxin-mu, Conotoxin-delta, Conotoxin-kappa, Cytotoxic necrotizing factor type I, oxynivalenol, Dermonecrotic toxin, Diacetoxyscirpenol, Diptheria toxin, EAST, Epsilon toxin, Equinatoxin II, Erythrogenic toxin, Exfoliatin toxin, Exotoxin A, Flavocetin, Hemolysin, Huwentoxin-I, Huwentoxin-II, Huwentoxin-IV, Iota toxin, Leukocydin F, Listeriorlysin O, LT toxin, Mastoparan, Nivalenol, Nodularin, Perfringolysin O, Perfringens enterotoxin, Pertussis toxin, Pneumolysin, Pyrogenic exotoxin, Ricin, Saxitosin, Scorpion toxin, Shiga, ST toxin, Staphylococcus enterotoxin, Streptolysin O, T-2 toxin, Tetanus toxin, Tetradotoxin, Toxic shock syndrome toxin, Toxin A, Toxin B and a radionuclide. In other embodiments, the toxin is selected from the group consisting of Anthrax toxin, Botulinum toxin (A), Botulinum toxin (B), Botulinum toxin (C), Botulinum toxin (D), Botulinum toxin (E), Botulinum toxin (F), Botulinum toxin (G), Ricin, Saxitosin, Staphylococcus enterotoxin and Tetradotoxin.

In still other embodiments, the toxin is a radionuclide such as a fission product, e.g., a lanthanide or an actinide such as americium-241, plutonium-239, plutonium-240, plutonium-238, uranium-238, uranium-235, europium-154, europium-155, cesium-137, strontium-90, iodine-131, iodine-125m, iodine-129, technetium-99m, neptunium-237, curium-244, rhenium-188, radium-228, radium-226, and cobalt-60.

In some embodiments, the particle has a paramagnetic core. Also in some embodiments, the core is encased in a compound selected from the group consisting of polystyrene, preferably monodisperse polystyrene, poly(lactic acid) and poly (lactic-glycolic acid).

In yet other embodiments of the method according to the invention, the particle is effectively coated with a polyalkylene glycol. An effective coating is a coating that is capable of preventing an immune response sufficient to render infeasible, clinically or economically, the use of a particle to remove a deleterious substance, such as a toxin, from an animal. Preferred polyalkylene glycols are polyethylene glycol and polypropylene glycol.

In still other embodiments, the particle used in the methods of the invention further comprises a specific binding partner for the substance, such as a toxin. Preferred specific binding partners include a receptor specific for a ligand, a ligand specific for a receptor, a ligand specific for a radionuclide, an antigen, a hapten and an antibody. In the present context, the term “specific” means that a compound or substance binds specifically to one, or at most a few (e.g., five), binding partners and distinguishes compounds that bind promiscuously or non-selectively. Preferably, the binding partner is a receptor for a ligand, or an antibody.

Yet other embodiments of the method are drawn to methods in which the organism from which the deleterious substance is removed is selected from the group consisting of a multicellular plant, a fish, an amphibian, a reptile and a mammal. Preferably, the organism is a human.

In practicing the methods according to the invention, the administering of the particle may be achieved using any technique known in the art, including injection, surgical implantation, catheterization, cannulation, transdermal delivery, oral delivery, anal delivery, spraying (e.g., intraluminal spraying), inhalation (assisted or not), and topical delivery. Further, particle administration may be continuous or intermittent, including the administration of a single bolus or dose, as well as multiple administrations using a schedule that is dependent on the context of the administration (e.g., nature of the substance; nature of the specific binding partner, if any; availability of medical facilities; and the like) and is determined using skills that are routine in the art.

Additionally, practice of the methods of the invention may involve the physical removal of the particle from a biological fluid, such as plasma, lymph, urine, or preferably, blood. The presence of particles in urine is not inconsistent with the design of the particles as compositions not readily cleared rapidly into urine because such particles would not be quantitatively and indefinitely excluded from the urine. The methods may also involve sequestration of the substance, e.g., in vivo, or alteration of the substance to effectively reduce or decrease the deleterious activity of the substance.

In another aspect of the invention, the magnetic particle, e.g., nanoparticle, is removed from the body or biological material such as a biological fluid (e.g., blood) by using a magnetic field gradient. In some embodiments, the magnetic field is an electromagnetic field, which may have a constant magnetic field gradient or a variable magnetic field gradient.

In accordance with this aspect of the invention, the method includes a particle removal step wherein the step of removing comprises: (a) circulating blood through a closed-loop catheter system in fluid communication with the bloodstream of the organism; (b) exposing the blood to a pre-defined magnetic field gradient, thereby impeding the flow of the particle in the blood; and (c) returning the blood to the organism.

The above-described methods of the invention may be used to remove exogenous substances, such as radionuclides arising from nuclear fallout, toxins released in an act of terror, or a drug overdose, among many other examples. In addition, the methods may be practiced on organisms harboring an endogenous substance that is deleterious, such as would be found in organisms (e.g., humans) having an autoimmune disease or disorder. Exemplary endogenous substances suitable for removal in accordance with the methods of the invention include an antibody (i.e., auto-antibody), a cancer cell, a DNA, a RNA, an immune substance, a cancer product, an abnormal cell and genetic material.

Yet another aspect of the invention provides a method for decreasing the deleterious activity of a substance in an organism by modulating the activity of the substance, comprising administering a biocompatible magnetic particle to an organism under conditions wherein the particle binds to the substance, wherein the particle has an in vivo half-life of at least fifteen minutes, and preferably at least thirty minutes, and wherein the bound substance exhibits detectably decreased deleterious activity, thereby decreasing the deleterious activity of the substance.

Still another aspect of the invention is a method of diagnosing a deleterious substance-induced condition in an organism comprising (a) administering a biocompatible magnetic particle to an organism under conditions wherein the particle binds to the substance, and wherein the particle has an in vivo half-life of at least fifteen minutes, and preferably at least thirty minutes; (b) removing the particle from the organism by exposing the particle to a magnetic field; and (c) identifying the deleterious substance, thereby diagnosing the condition. The deleterious substance may be removed while attached to the particle, or detached therefrom. A variety of particles are contemplated for this aspect of the invention, including the use of bi- and multi-modal particles (particles having a plurality of distinct binding partners attached), alone or in combination with other, distinct, multi-modal particles. In such embodiments, one or more particles is expected to specifically bind to a deleterious substance, such as a toxin, without prior knowledge of the identity of that substance.

In another aspect of the methods and systems of the invention, a single medication or diagnostic substance is bound to the magnetic sphere surface or incorporated into the sphere matrix. The invention contemplates a variety of other formats for medication- or diagnostic substance-particle attachment, such as a plurality of distinct medications and/or diagnostics bound to a particle, a mixture or collection of particles collectively bearing a plurality of medication or diagnostic substances, wherein each particle is attached to one type of medication and/or diagnostic (a mixture of such particles refers to co-administration of the particles, whereas a collection of such particles does not require co-administration), and a plurality of particles, wherein each particle is attached to a plurality of distinct medications and/or diagnostics. For example, an alternate embodiment provides a ‘cocktail’ of particles where a single injection of functionalized spheres contains magnetic particles, each containing several different medications and/or diagnostics.

In yet other alternative embodiments, non-specific surface interaction between the magnetic particles and an implanted magnetizable stent or seed are contemplated. Once injected, e.g., into the bloodstream, the magnetic particles flow through the blood and bind to the magnetized stent or seed. Magnetic attraction of the medication or diagnostic substance which is coupled to the magnetic spheres and the implanted stent or seed magnetized by an external magnetic source will lead secondarily to either chemical binding of the medication or diagnostic substance at the stent or seed site or alternatively to simple deposition of medication or diagnostic substance in the tissue or organ surrounding the stent/seed.

Functionalized magnetic particles, e.g., spheres, can be removed from the bloodstream using a technique called extracorporeal magnetic separation. This technique uses a device that consists of a short, small diameter, non-clotting loop circulating blood directly from an artery, or vein, back to a vein (artery-vein circuit or vein-vein circuit). In midsection, the tube branches into several smaller tubes or an array of smaller tubes that penetrate a magnetic field gradient contained in a housing. The magnetic field traps and sequesters circulating magnetic spheres in a specific section of the tube. Blood free of magnetic spheres is then returned to the body by the tube. The sequestration unit can contain either a permanent magnet or an electromagnet activated by, e.g., a small battery source. The blood-circulating tubes may pass under, over, or through the magnet to create a field gradient across the inner diameter of the tubes.

Another aspect of the invention provides a kit comprising a compound attached to a magnetic particle as described herein and a set of instructions for administration of said compound to treat or diagnose a disease, disorder or condition in an organism in need, or to remove, inhibit or inactivate a deleterious substance, such as a toxin.

Other aspects and advantages of the invention will be apparent upon consideration of the following drawing and detailed description.

### BRIEF DESCRIPTION OF THE DRAWING

FIG. 1 shows a graphic representation of in vitro sequestration of a biotinylated enzyme from simple fluids and whole rat blood under static and dynamic flow conditions.

FIG. 2 shows the streamlines of single drug carriers, or MPs, resulting from simulations using Femlab, a commercially available magnetic field-fluid flow model, with flow moving from left to right, through the 8-loop stent and under a field of 1.0 T perpendicular to both the plane of the figure and the blood flow. The radius of the stent cross sectional area was 2.5 mm, velocity in the upstream vessel was 0.8 ms⁻¹, and the angle of the field was 90°. The single drug carriers consisted of non-porous spheres of 2.0 mm in radius that contained 80% (w/w) magnetite. The collection efficiency for this case is 26%.

FIG. 3 provides a graphic illustration of the orientation of the magnetic field vector, Hₒ, blood flow velocity Uₒ, and stent wire segment. To better understand the capture of magnetic particles by a magnetizable stent, a computational model was developed and the parameters were varied to determine the sensitivity on the capture cross section (y-axis, distance between particle and stent where capture is possible). The stent can be modeled as a net made of metal wires. Thus, a piece of wire can be studied instead of the entire coil to simplify analysis. In practice, the external magnetic field H₀ can easily be kept perpendicular to the blood flow U₀. Thus, the position between H₀, U₀ and wire could be simplified as in FIG. 2.

FIG. 4 provides a conceptual model describing the capture of magnetic microspheres by a magnetizable wire. The dimensionless capture cross-section is defined as follows (Ebner and Ritter, 2001)

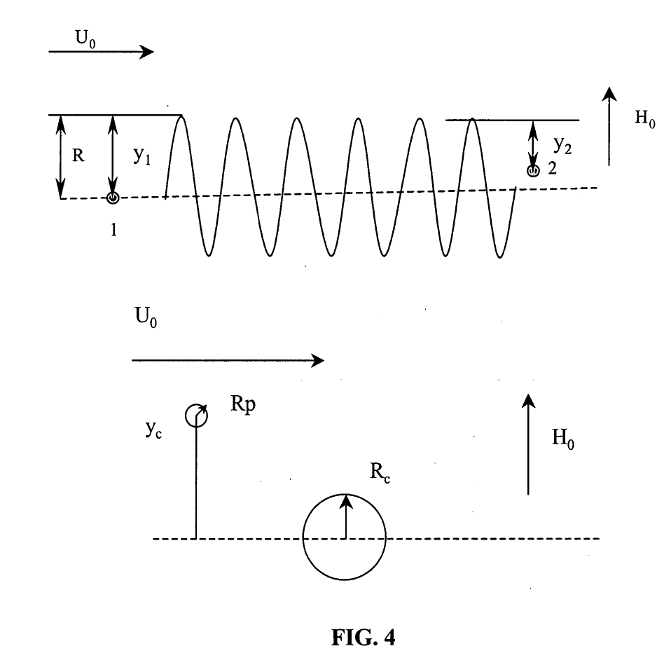

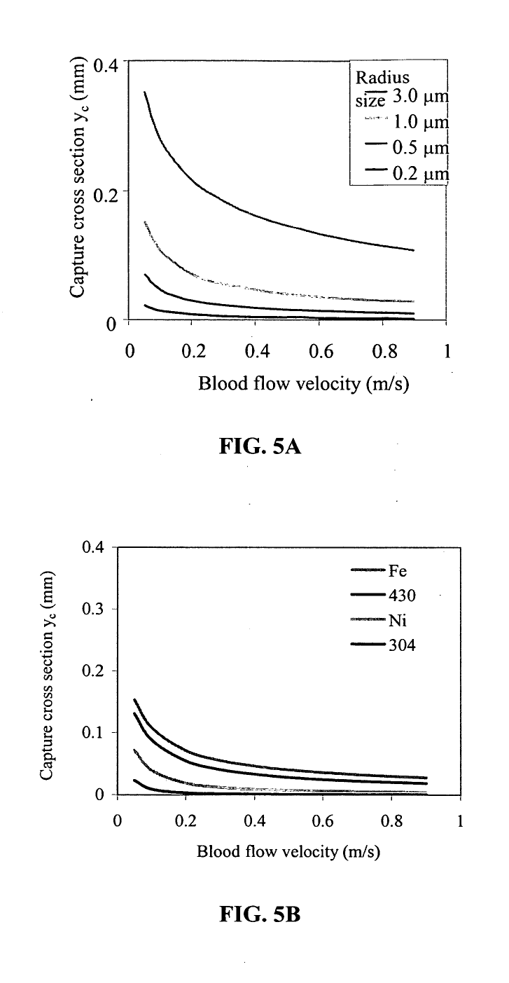

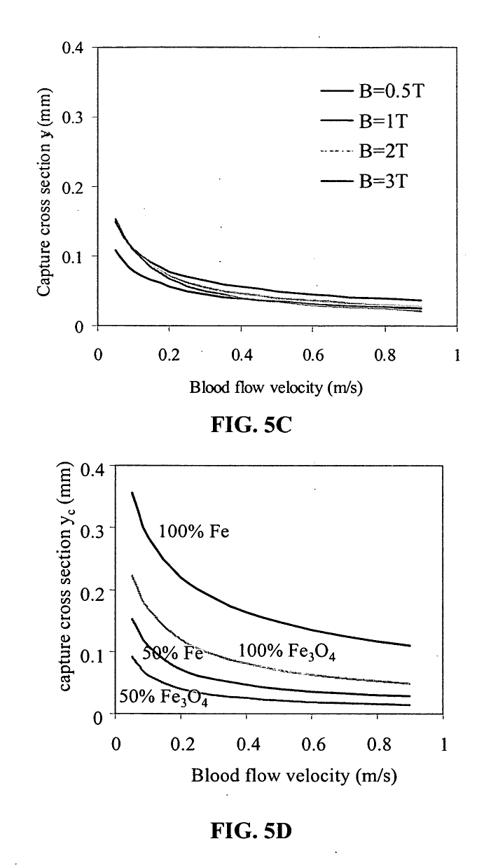

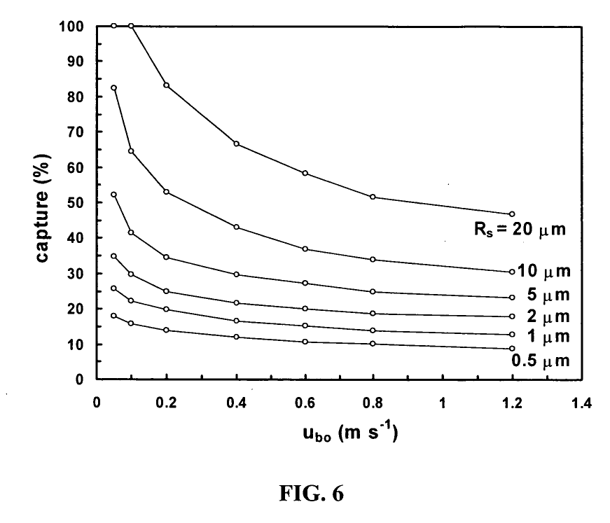

$$λ = \frac{{y}_{c}}{{R}_{c}} , \qquad ( 1 )$$

where y_{c} is the capture cross-section and R_{c} is the radius of the wire. The conceptual model is based on a coiled stent according to the invention. R is the radius of expanded stent; y₁ and y₂ are the distances from the center of a stent coil wire to the center of particle 1 and particle 2, respectively (FIG. 4, top). H₀ is magnetic field; U₀ is blood flow; capture cross-sections are y_{c1} and y_{c2}, respectively, which are the maximum perpendicular distance that particle 1 and particle 2, respectively, could pass from the center of the wire and still be captured by the magnetized wire (FIG. 4, bottom).

FIG. 5 illustrates the capture cross section ymax, for magnetic spheres in fluid flow fields [radius of stent wire=0.0625 mm; radius of particle=1 mm; magnetic core of particles=Fe 50% (w/w); magnetic field=2 T; velocity of flow=20 cm/s; density of the particle polymer=0.95 g/ml; density of the fluid (blood)=1.04 g/ml; viscosity of the fluid (blood)=0.003 Pa; ferromagnetic stent material=Fe.]

FIG. 6 shows the results of in vivo Femlab simulation experiments, suggesting that magnetic particles may aggregate during flow, with aggregated particles being more efficiently captured. Aggregates the size of 20 μm were expected to be captured at 50% efficiency in high flow arteries (u_{bo}=100 cm/s). Magnetic capture of magnetic microspheres and microsphere agglomerations by a coil of eight magnetizable wires. Applied field=0.5 T, carriers with radii=0.5, 1.0 and 2.0 μm represented single particles while the carriers with a radius=5.0, 10.0 and 20.0 μm represented porous carriers (porosity=0.4, i.e., agglomerates of single particles).

### DETAILED DESCRIPTION OF THE INVENTION

The invention provides systems and associated methods and materials for effectively delivering, in one or more doses, a variety of medications, drugs, and diagnostics to a predefined tissue, organ, organ system, or body region in an organism. The delivery is selective and minimally invasive, and, in the case of animals such as humans, delivery typically takes advantage of the bloodstream as a conduit for effective delivery of the medicated or diagnostic substance. Invasive procedures may be minimized by using a magnetizable stent and/or an implanted, magnetizable seed in combination with biostabilized and/or compatible magnetic particles (MPs) in the size range of nanometers and micrometers that are preferably injected directly into the bloodstream, either intravenously or intraarterially. Magnetic sequestration of the particles at the stent site provides a basis for the subsequent controlled release of the particles to provide a variety of dosing schedules resulting from one or a few invasive procedures. Specific binding partners such as medications (e.g., platelet aggregation inhibitors or thrombolytics (clot busters), radionuclide therapeutics, antibodies, chemotherapeutics, receptor mimetics and others) attached to the MP surface or encapsulated within the MP are selectively delivered to the magnetizable stent and its surrounding regions/tissues. To do so, the medicated or otherwise treated, biostabilized magnetic particles circulate after injection throughout the bloodstream and the MP will, over time, reach the area of the magnetizable stent or seed and become trapped by the magnetic force generated by the stent or seed. The stent or seed, in turn, will only be made temporarily magnetizable using an external magnetic force of appropriate strength and duration. Drug-stent delivery systems according to the invention are based on a combination of a) a magnetizable metallic stent or seed device, suitable for implantation, and b) targeted medication and/or diagnostic drugs associated with magnetic micro- or nanospheres that are suitable for administration to an organism in need. These spheres are smaller than red blood cells and can be as small as about 100 nanometers in diameter. They are composed of biodegradable polymers such as poly(lactic-acid) and contain the drug on the surface or encapsulated within the spherical core. Magnetic nanophases (typically iron oxides) are encapsulated inside the sphere during synthesis and impart the magnetic responsiveness of the sphere. In the current invention these spheres are injected into the blood, sequestered from the bloodstream at the stent site, and subsequently delivered into the surrounding tissue by manipulation of an external magnetic field generator (e.g., electromagnet or permanent magnet).

The invention further provides systems and associated methods for removing a variety of deleterious substances and, in particular, toxins present in an organism. The removal is selective and minimally invasive, and, in the case of animals such as humans, removal typically takes advantage of the bloodstream as a conduit for effective removal of the deleterious substance. Biostabilized and/or compatible magnetic particles (MPs) in the size range of nanometers and micrometers are preferably injected directly into the bloodstream. Specific binding partners such as anti-toxins (e.g., antibodies, radionuclide extractants, chelators, ligands, receptor mimetics and others) attached to the MP surface selectively capture deleterious substances such as toxins and form MP-toxin complexes. Alternatively, PEGylated MPs are used to bind non-specifically to toxins and various other blood-borne substances in the bloodstream, followed by extraction from the blood, as described below. After an appropriate circulation time, a combined catheter/tubing and magnetic filtration system is connected to an artery or vein where the MP-toxin complexes are magnetically separated from the blood. To facilitate a more thorough understanding of the invention, the following term definitions are provided.

A “medication or diagnostic substance” is any compound or composition, whether produced by a living organism (foreign to the host or the host itself) or through artificial means, such as by chemical synthesis or ex vivo biochemical synthesis, that is capable of causing a therapeutic or diagnostic effect when introduced into an organism, e.g., via the bloodstream. Such substances may be capable of interacting with specific regions of a body, such as a tissue, or its effects may be localized as a result of the influence of a magnetizable stent to which it is physically attracted. Such substances are useful in treating or diagnosing a status or condition of a cell, tissue, organ, organ system, or body as a whole, resulting in a detectable improvement in the health of an organism.

A “deleterious substance” is any substance that impairs the health of an organism, and includes substances of extracorporeal origin, i.e., exogenous substances (e.g., environmental biohazards and toxins), or endogenous substances (e.g., auto-antibodies, such as are found in auto-immune diseases or conditions). Deleterious substances generally, and toxins more particularly, are exemplified below.

“Biocompatible,” as used in the context of a “biocompatible magnetic particle,” means a magnetic particle that is able to exist in vivo without inducing a deleterious host reaction, e.g., a deleterious immune response, that would significantly impair the capacity of the magnetic particle to function in the effective delivery of a medication or diagnostic substance or in the removal of a deleterious substance. Biocompatible particles, medications, or diagnostic substances may or may not be harmful to the organism in that certain particles, medication or substances may be designed to harm a cell, tissue, organ, or organ system, or to exert a systemically harmful effect, such as in anti-cancer therapies. It is understood that the effective delivery is to the affected organism as a whole, or the organ system, organ, tissue or cell in need.

“Magnetic particle” is given its ordinary and accustomed meaning of a particle exhibiting the defining characteristic of a magnetic moment when exposed to a magnetic field of at least a particular strength. Typically, a magnetic particle exhibits a moment sufficient to permit effective control of the spatial location of the particle in the presence of a given magnetic field. A “magnetic sphere” is one type of “magnetic particle” according to the invention.

“Magnetic field” is given its ordinary and accustomed meaning in the art and includes permanent, variable, or transient magnetic fields, and constant or gradient magnetic fields.

“Magnetic attraction” is given its ordinary and accustomed meaning of a force, capable of acting at a distance through a magnetic field that is capable of urging at least two masses (e.g., particles) towards, or away from, each other. Either or both of the masses may be paramagnetic or ferromagnetic substances, such as a magnetizable stent or seed, or an MP. Magnetic attraction may or may not be associated with simultaneous or consecutive binding of the medication or diagnostic substance delivered by an MP and the stent or seed or surrounding tissue or organ system. Non-magnetic binding of the medication or diagnostic substance can be achieved using covalent or non-covalent bonding (e.g., hydrogen bonding, van der Waals forces), it may be permanent, and it may be either specific or non-specific. To be effective, the magnetic attraction between the stent or seed and a magnetic particle (whether bearing a specific binding partner or not) must sufficiently overcome all forces opposing the magnetic attraction, such as flow shear stress and others as described herein.

A “stent” is given its ordinary and accustomed meaning in the art of a mechanical hollow vessel segment, typically cylindrical, that is generally used to maintain vessel patency in an organism in need. The stent may be of any length, of any mean diameter, of any wall thickness, and tapered, depending on the context of its usage, as would be known in the art. A “magnetizable stent” is any stent capable of temporary, or permanent, magnetization. Magnetizable stents may be made of any material known in the art to be capable of temporary or permanent magnetization, such as a paramagnetic metal or metal alloy. Preferred magnetizable stents are made of one or more biocompatible materials, including materials rendered biocompatible by any surface coating or treatment known in the art to provide biocompatibility.

A “seed” is a magnetizable material for releasably, or permanently, sequestering magnetic particles containing a medication, diagnostic agent or deleterious substance. The seed is analogous to the magnetizable stent described above, except for the absence of any requirement to have a lumen compatible with non-occlusive vessel placement. A seed can be made of any of a variety of materials, alone or in combination, provided that the seed retains the capacity of being magnetizable and can be rendered biocompatible. The form and dimension of a seed can vary, depending upon the context of a particular usage, as would be understood in the art. These magnetizable seeds are contemplated as useful in controlling the localization and/or delivery of coated MPs in non-vessel locations (preferred) and vessel locations within a body.

“Conditions suitable for binding” is given its ordinary and accustomed meaning of suitable values for those variables, e.g., temperature, pH, reagent concentration(s), time, and the like, capable of influencing the capacity of specific binding partners to effectively associate, or bind, as would be understood in the art. The bloodstream of a warm-blooded animal is expressly defined as providing conditions suitable for binding.

“Conditions suitable for magnetic attraction” is given its ordinary and accustomed meaning of suitable values for those variables, e.g., magnetic force, temperature, pH, reagent concentration(s), time, and the like, capable of influencing the capacity of specific binding partners to effectively associate, or bind, as would be understood in the art. The stent or seed implanted into a warm-blooded animal is expressly defined as providing conditions suitable for magnetic attraction for medicated or diagnostic MPs.

“Bind” is given its ordinary and accustomed meaning of effective association between at least two distinct compounds or compositions, such as an antigen and an antibody specifically recognizing that antigen. Binding can be achieved using covalent or non-covalent bonding (e.g., hydrogen bonding, van der Waals forces), may be permanent, and may be specific or non-specific. Preferably, the bond or association between a deleterious substance and a magnetic particle (whether bearing a specific binding partner or not) survives sufficiently to permit manipulation of the particle, resulting in effective manipulation of the deleterious substance.

“Binding partner” is given the meaning it has acquired in the art as a member of a group, typically a pair, of compounds that have the capacity to specifically bind each other. Binding partners include, but are not limited to, antigen-antibody pairs, carbohydrate-lectin pairs, proteins that specifically interact with a compound such as streptavidin-biotin, ligand-receptor pairs, chemical ligand-radionuclide pairs, and the like.

“In vivo half-life” is given the meaning it has acquired in the art of the time taken to reduce the in vivo amount of a compound or composition, or its relevant activity, to one half of its original value, as measured by monitoring a defining property of that compound or composition, such as physical integrity, activity, and the like.

“Uptake” means effective loss, partially or completely, of the activity of a substance such as a medication, from the circulating blood to an organ system, organ, tissue or cell in need. Examples of uptake include physical removal, sequestration, or modulation leading to reduced blood concentration. In the context of a medication or diagnostic substance, “uptake” means physical removal, sequestration or localization, or modulation that effectively reduces the presence of the substance within the bloodstream.

“Removing” means effectively withdrawing, such as by physical removal, sequestration, or modulation, leading to reduced activity. In the context of a deleterious substance, “removal” means physical removal, sequestration or localization, or modulation that effectively reduces the deleterious property of the substance.

“Toxin” means a poison, whether produced by a living organism (foreign to the host or the host itself) or through artificial means, such as by chemical synthesis or ex vivo biochemical synthesis. A toxin is capable of causing damage or disease when exposed to a body tissue but it is often also capable of inducing a neutralizing antibody or antitoxin; such poisonous substances can also be chemicals and radioactive materials causing damage or disease to humans and/or other animals or plants. Toxins are exemplified hereinbelow.

“Effectively coated” in the context of an “effectively coated magnetic particle,” or sphere, means that the exterior surface of a particle (sphere) is associated with a compound or composition (the coating) at sufficient surface density to detectably alter a host organism's defense response (e.g., immune response) relative to a host organism's defense response to an uncoated particle (sphere). Typically, an effective coating detectably lowers a host organism's defense response, increasing the in vivo survival time of a coated particle relative to an uncoated particle.

“Biological fluid” is given its ordinary and accustomed meaning of any biological substance in a fluid state. Examples of biological fluids include blood, plasma, lymph, aqueous humor, plant sap, and the like.

“Modulating the activity” means affecting the activity relative to a control state, and includes an increase or a decrease in activity relative to that control, as would be understood in the art. A “detectably decreased deleterious activity” is an example of a modulated activity and means that the deleterious property of a compound or composition has been reduced to a detectable extent.

A “chemical” is a substance with a definite molecular composition that is produced by or used in a chemical process.

“Medication” and “drug” refer to a pharmacologically active substance typically used in medicine to treat, or induce a process of treating, a condition or disease in an organ system, organ, tissue, cell(s), or body as a whole of an organism. The “medication,” “drug,” or “diagnostic substance” may be administered via the bloodstream. Such a substance is typically capable of interacting with at least one specific region of an organism, such as a region comprising a metallic surface of an implanted stent.

“Genetic structure” is a hereditary unit consisting of a sequence of DNA or RNA, typically encoding an RNA or a protein and including the coding region and associated regulatory elements, such as promoters, operators, enhancers, terminators and untranslated regions.

A “protein” is any of a group of complex organic macromolecules that contain carbon, hydrogen, oxygen, nitrogen, and usually sulfur and is composed of one or more chains of amino acids. Proteins are fundamental components of all living cells, as exemplified by enzymes, hormones, and antibodies.

Informed by these definitions, the invention stands in contrast to conventional systems and methods for delivering or removing a compound (e.g., a medication or medicament, diagnostic substance or agent, or a deleterious substance) to, or from, an organism such as a human in using magnetically controllable MPs in an in vivo environment, frequently in conjunction with a magnetizable stent or seed. The methods and systems of the invention provide at least one of the following advantages: (A) specificity of a medication or diagnostic substance wherein only substances attracted magnetically to the magnetizable stent surface will be trapped at the stent site; (B) targeted delivery by removal of the medication or diagnostic substance from the bloodstream and deposition at the stent site and into the stent-surrounding tissue where several important advantages exist: (i) the medication or diagnostic substance can be conveniently administered, e.g., injected or inhaled, at a body site distant from the actual stent, for example, systemically (intravenously or intraarterially), into the brain-spinal cord fluid (cerebrovascular fluid; intrathecally), or directly into an organ or tissue; (ii) because this targeted delivery system depletes concentrations in the circulating blood over time it can be used repeatedly, that is, the medication or diagnostic substance injection can be arranged to fit various treatment schedules; and (iii) quantification of removed medication or diagnostic substance permits direct estimation of the efficiency of stent-mediated medication or diagnostic substance removal, an estimate of required therapy duration; for example, the time and strength of external magnetization needed to concentrate all magnetic medication or diagnostic substance at the stent site; (C) tissue delivery of the injected medication or diagnostic substance is facilitated by a magnetic field gradient extending between the magnetizable stent and the external magnet as this field gradient will first concentrate form the bloodstream all, or most, of the injected magnetic spheres at the stent site and subsequently urge the medicated spheres from the stent site towards the direction of the strongest external magnetic field gradient and therefore into the stent-surrounding organ or tissue; this becomes especially useful when repeated targeted treatment of an organ or tissue system is required; (D) non-toxicity where nanoparticles remaining within the body are metabolized physiologically or remain undegraded without adverse effects; (E) circulating magnetic spheres functionalized with medication or diagnostic substances which are not trapped or concentrated or not anymore needed at the stent site, i.e., after a predefined time or treatment interval, can subsequently be efficiently removed from the bloodstream, if necessary, wherein a specially designed external magnetic filtration unit utilizing extracorporeal circulation of blood allows high efficiency removal of magnetic particles from the bloodstream; (F) convenience and relative non-invasiveness of therapy or diagnosis wherein a magnetic sphere is functionalized with a medication or a diagnostic substance that can be injected at any location, i.e., other than in a hospital, which has both the external magnetic unit and the injected magnetic spheres available, thereby simplifying the chronic treatment of patients. The initial placement of the magnetizable stent or seed is typically invasive, which requires a hospital setting for a single routine surgical or endovascular procedure with placement of the stent or seed within the target region of the body; (G) convenience, wherein a two-step drug or diagnostic substance delivery can be simplified for large-scale implementation aided by, e.g., non-verbal visual guides (i.e., graphic instruction as to placement of the external magnet at a predefined body site) followed by simple needle insertion and injection of the medication or diagnostic substance; (H) safety in that there are significantly reduced risks of the injected medication or diagnostic substance causing systemic adverse effects as the treatment is (i) targeted to a specific body region, markedly reducing long-term exposure and deposition of the medication or diagnostic substance or substances to other body regions such as the liver, kidney, central nervous system, and the like, and (ii) the magnetic particles can be specifically, quantitatively and actively removed from the bloodstream using an external magnetic filtration unit; and (I) repeatability insofar as re-treatments or re-diagnosis can conveniently be performed by repeated injection of the medication or diagnostic substance(s) using an external magnet unit as often as medically indicated.

Another aspect of the invention provides methods and systems for removing deleterious substances from an organism that stand in contrast to conventional hemodialysis and other, less common, detoxification systems. These methods and systems of the invention provide at least one of the following advantages: (A) specificity of toxin removal wherein only substances binding specifically to the magnetic particle-ligand surface will be removed; (B) removal of toxins from the body, thereby providing the advantages of: (i) reduced likelihood of rebound toxicemia caused by the dissociation of toxin-antitoxin complexes because the complexes are removed from the body; (ii) the secondary depletion of toxin stores in tissues due to equilibrium driven shifts of such stores to the bloodstream, from which the toxins are removed on a continuous or punctuated schedule; and (iii) quantification of removed toxin permitting direct estimation of toxin removal efficiency, thereby providing a basis for estimating the required duration of therapy; as well as an opportunity to perform additional analyses on the removed toxin to investigate potential foul play (e.g., bio-forensics), to improve anti-toxin therapy, and the like; (C) toxin cleansing, wherein toxin binding is facilitated by using particles having large anti-toxin binding capacities to permit the use of lower affinity anti-toxins (e.g., toxin binding agents); (D) non-toxicity where magnetic particles (e.g., nanoparticles) remaining within the body are metabolized physiologically or remain undegraded without adverse effects; (E) efficiency of removal, wherein specially designed magnetic filtration units facilitate the high-efficiency removal of magnetic particles; (F) portability, wherein the magnetic filtration units and injectable particles can be used in the field, e.g., on a battlefield or carried by emergency response vehicles to various field sites because of their relatively light weight and compact design; (G) convenience, wherein a two-step detoxification can be simplified for large-scale implementation aided by, e.g., non-verbal visual guides (i.e., graphic instruction as to the site for administering magnetic particles, e.g., by injection, as well as a body site for establishing fluid communication with an external magnetic filtration unit, whether a miniaturized portable unit or a relatively larger hospital-based unit is employed; (H) safety in that there are significantly reduced risks of disease transmission (unlike antibody-based treatments, blood transfusions, and other approaches reliant on biological materials); and little or no blood loss, with closed loop, pre-heparinized and pre-sterilized, single-use systems avoiding blood contamination and allowing self- or helper-applied usage by non-medical personnel while preserving samples of removed toxins for further investigation; and (I) repeatability insofar as re-exposures to biohazards or re-accumulation of toxin from body tissue stores can conveniently be treated with single- or multi-session punctuated toxin removal procedures, or the removal can be continuous and for varying time periods, as would be determinable by those of skill in the art.

Magnetizable stents according to the invention, whether used to facilitate the administration of a medication-containing MP or a diagnostic agent-containing MP, or to facilitate the removal of a deleterious substance in conjunction with an MP, are typically located within the lumen of a vessel of an organism. The placement of such stents are accomplished through routine surgical procedures well known in the art, such as conventional surgical intervention, laparoscopic placement, catheter-mediated placement, dermal incision and transdermal placement, image-guided placement (e.g., CT-guided, fluoroscopic guidance), stereotactic placement (e.g., seed placement into brain parenchyma), endoscopically guided placement (e.g., bronchoscopy, gastroscopy), and the like. The location of these placements include intraarterial, intravenous, respiratory, intraintestinal, intrabiliary, intraurinary (including intraurethral, and intravesicular), intrarenal, and intragenital tract placement.

By way of non-limiting example, a magnetizable coronary stent is loaded, or coated, with a magnetic particle attached to a medication known in the art to inhibit or prevent stent overgrowth, or to inhibit, prevent or treat coronary artery thrombosis. Alternatively, an uncoated stent is placed in a coronary artery and the medication-containing MPs are administered conventionally, with the MPs magnetically sequestered at the stent site. Even in the case of pre-loaded stents, magnetic sequestration is expected to be useful in containing the medication at the site of treatment. Further, magnetizable stents, whether pre-loaded with a medication-containing MP or not, are expected to be useful in providing medication(s) known in the art that support myocardial function and/or in facilitating the placement of additional magnetizable devices during interventional or surgical procedures.

A second, non-limiting example is an intrahepatic magnetizable stent providing patency to a hepato-biliary vessel, and facilitating the localized delivery of a medication or diagnostic agent to the hepato-biliary system. An intraheptic magnetizable stent is expected to be useful in treating a variety of hepato-biliary diseases including, e.g., a hepatic tumor or a biliary tumor, using a radiological or chemical medication.

Beyond placement in the hepato-biliary system, other non-vascular placements of a magnetizable stent include placement in the respiratory, genital or urinary systems. Such stents are useful in maintaining patency (e.g., maintaining bronchial patency in the presence of a peri-bronchial tumor, maintaining patency in the presence of a genital or urinary tract tumor), providing for the localized release of a medication or diagnostic agent from a pre-loaded stent, and in localizing a medication or diagnostic agent associated with an MP that is administered conventionally (e.g., injection by any known route, including intraarterial or intravenous administration as well as luminal injection; luminal spraying, inhalation, transdernal delivery). Temporary modulation of the magnetic interaction between the stent and the MP containing a medication and/or diagnostic agent can be effected by an external magnetic field generator, resulting in local delivery of the medication or diagnostic agent to the peri-stent tissue, organ or organ system.

The invention also comprehends the placement of a magnetizable seed in the intraluminal locations described above for stents, particularly where such seeds will not appreciably interfere with the passage of materials through the vessel in a manner characteristic of a healthy organism. The magnetizable stents and seeds, preferably the seeds, may also be located outside a vessel in an organism, e.g., in the intercellular space of a tissue in need of treatment, diagnosis, or deleterious substance removal, or, for example within the respiratory system (intrapulmonary or intrabronchial placement). Such seed placements need not be in direct contact with an element of the vasculature. Generally, a seed according to the invention is contemplated for placement in any body region including, but not limited to, a body cavity, a tumor, a firm or soft organ or tissue, parenchyma, or bone. Further, the techniques described herein for placement of a magnetizable stent are also suitable for placement of a magnetizable seed. The invention further contemplates a magnetizable stent or seed partially or completely coated with an angiogenic or vasculogenic agent (e.g., vascular endothelial cell growth factor) to facilitate development of vessels in the vicinity of the stent or seed placement, including those placements that do not result in direct contact between the seed and an element of the vasculature.

A non-limiting example of a method of treatment using a magnetizable seed and a medication-containing MP according to the invention is the treatment of a brain tumor. A magnetizable seed is placed intraparenchymally, e.g., during tumor excision, within tissue of the brain. In one embodiment, an MP associated with a known anti-cancer medication is injected intravenously and localized to the seed using magnetic force. Repeated injections ensure that sufficient medication reaches the site of the brain tumor without running the risk of administering toxic dosages. Further manipulation of a magnetic field is contemplated for the localized release of sequestered MPs containing the medication, consistent with clinical indications.

Even in their simplest forms, systems of in vivo detoxification using biostabilized magnetic particles have diverse applications. Examples include, but are not limited to, 1) diagnosis of exposure to toxin(s), toxin precursor(s), and diagnosis of a disease; 2) removal of toxins (e.g., warfare toxins) and secondary toxins, including biological, chemical, and radioactive poisons; 3) treatment of drug and medication overdoses; 4) and treatment of acute and chronic medical illness, including use in chronic diseases or continuous or repeated toxin exposure, as repeated particle-based detoxification is contemplated. Repeated chronic detoxifications can lower toxic tissue stores over time and achieve long-term detoxification.

The systems of non-invasive, yet targeted, in vivo therapy or diagnosis with biostabilized magnetic spheres or particles has diverse applications. Examples include, but are not limited to, 1) diagnosis of a biological process or state such as recognition and delineation of a disease state; 2) therapy of a biological process or state such as chemotherapeutic treatment of a cancerous condition or re-opening of an acutely occluded stent located within a blood vessel; 3) simultaneous monitoring and treating of a stent-surrounding tissue or organ system by combining magnetic spheres suitable for both diagnosis and treatment; and 4) treatment of acute as well as chronic medical illnesses, such as an acute occlusion of a stent by a blood clot, or chronically treating a cancerous condition by placement of a magnetizable stent in the vicinity thereof, followed by magnetically controlled therapeutic administrations from the stent. Repeated chronic treatment with functionalized magnetic particles, such as spheres, can significantly lower the long-term toxic effects to body regions other than a target site (e.g., cell, tissue, organ, or organ system). The ease of administration of the magnetic spheres is expected to make them useful as a diagnostic and/or therapeutic tool.

The methods of the invention provide for systemic injection, targeted (that is, focal) medication or diagnostic substance delivery with effective pharmacokinetics and dynamics at the target site as well as making possible the specific and quantitative removal of any medication or diagnostic substance from the bloodstream. This system differs from previous techniques because it employs a different principle of target diagnosis and/or treatment consisting of three basic steps: injection of functionalized, biocompatible, freely circulating magnetic particles, e.g., nanospheres, into the bloodstream; binding of the magnetic spheres to the temporarily or permanently magnetized stent or seed with or without the use of an external magnetic field; and controlled release of the medication/diagnostic agent-containing particles from the stent or seed.

In other embodiments, the invention provides the advantages of a simple, low cost; versatile and effective approach to detoxification. The ease of administration of the particles is expected to make them useful as a diagnostic and/or therapeutic tool, i.e., for non-medical personnel (e.g., military personnel, first-response units, civilians). Further, the methods provide a concentrated form of the removed deleterious substance in the form of a concentrated analyte, suitable for bioassay, chemical exposure assay, radiological assay, or mass screening, facilitating precise identification and/or characterization of the deleterious substance and providing information useful in estimating re-injection dosages of particles to complete detoxification or to determine the need for other, conventional, treatments. In addition, the methods of the invention are amenable to the removal, sequestration or effective inactivation of a wide variety of deleterious substances, e.g., biochemical, infective, and radioactive toxins, antibodies, cells, and other particles, having various biological, chemical and physical properties. Also, the methods are expected to exhibit relatively high toxin specificities and removal efficiencies. In various embodiments, the invention also provides for portability, allowing implementation of the methods of the invention in a variety of settings, including emergency and non-emergency scenarios, medical and in-field applications, and self- or helper-system applications.

The methods of the invention provide for the removal, sequestration or effective conversion to a non-deleterious state of various biological, chemical, and radioactive substances, preferably from the bloodstream, generally referred to herein as toxins. This system differs from previous techniques because it employs a different principle of detoxification consisting of three basic steps: injection of functionalized, biocompatible, freely circulating magnetic particles, e.g., nanoparticles, into the bloodstream; binding of the blood toxin to magnetic particles to generate composites; and extracorporeal separation of the composites by a sufficiently strong magnetic field gradient.

Self-applications are preferred in emergent situations where the exposed individual utilizes a ready-to-use detoxification package or kit to perform the following steps: a) needle-catheter placement for vascular venous or arterial access, b) self-injection of antitoxin-loaded magnetic particles from prefilled syringes, and c) connection and use of a portable detoxification device for a predefined time-period. Helper-applications include a variety of modifications to the self-application, utilizing one or more assistants to perform vascular access, injection of particles, and detoxification in the field, mobile medical unit, or hospital.

Other aspects of the invention are drawn to magnetizable stent-based methods and systems for the delivery of a medication, such as a therapeutic or a drug, or a diagnostic substance based on a combination of a) an implanted magnetizable stent or seed device, typically metallic, and b) targeted medication and/or diagnostic drugs injected intravenously, or intraarterially based on medicated, magnetic micro- or nanoparticles, such as spheres. These spheres are smaller than red blood cells and can be as small as about 100 nanometers in diameter. They are typically composed of biodegradable polymers such as poly(lactic-acid) and contain the drug on the surface or encapsulated within the spherical core. Magnetic nanophases (typically iron oxides) are encapsulated inside the sphere during synthesis and impart magnetic responsiveness to the sphere. In the methods and systems of the invention, these particles may be injected into the blood, sequestered from the bloodstream and delivered to the stent, with subsequent delivery of the particles into the surrounding tissue. Delivery is conveniently effected by the influence of an appropriate magnetic field, or flux therein, preferably under the control of an external magnetic field generator, such as an electromagnet or permanent magnet.

Biocompatible Magnetic Particles

Magnetic particles (e.g., nanoparticles) of optimal size avoid obstructing capillary blood flow and immediate vascular clearance, with surface properties that prolong vascular circulation. Specifically, the magnetic particles are preferably between 100 and 5000 nanometers (nm); also preferred are magnetic particles between 400 nm and 3000 nm. The surface charge is preferably near neutral (<20 mV, measured by zeta-potential) due to the surface coating (e.g., PEGs) in order to increase blood circulation time and minimize bioclearance by opsonization and phagocytosis.

The limits set by physical removal of particles flowing in the blood are rather broad. To maximize surface receptor density, particles with sufficiently high surface area-to-volume ratios are desired. High surface-area-to-volume ratio particles would imply using the smallest particle possible, or those <100 nm. However, as one reduces the particle size, it becomes more difficult to sustain the magnetite content or magnetic moment. In other words, the magnetic moment drops at a rate faster than would be expected based on the reduction in particle volume as the diameter is reduced. Successful separation of 400 nm polystyrene magnetic particles from blood has been achieved, even though the magnetic moment of these particles was low (i.e., 3 emu/g). Thus, a particle in the range of 100-5000 nm is preferred.

The force on a magnetic sphere is defined by \vec{F}=μₒ\vec{B}∇\vec{B} where \vec{B} is the magnetic field strength, ∇\vec{B} is the gradient in the field, μₒ is the magnetization of the sphere and is a function of the magnetic nanophases inside the polymer sphere. To cause the compound-loaded spheres to deviate from normal blood flow patterns, the magnetic force between the stent and the spheres is used. A magnetizable stent may comprise an array of wires that create high local magnetic field gradients when immersed in a magnetic field (uniform as in an MRI unit or not). Strong forces are generated causing the magnetic spheres to deflect towards, and attach to, the magnetized wires of the stent. A stronger magnetic field produced by the magnetized stent and external magnetic field will help not only in the sequestration of magnetic particles from blood flow but also in the delivery of these particles into the surrounding tissue (i.e. extravasation).

Any techniques known in the art may be used to apply a coating (e.g., a PEG) to the particle surface. A hydrophilic coating of the magnetic particles is desirable to facilitate biostabilization and RES avoidance. Typically, polyethylene glycols (PEG) and derivatives are used. There are two general methods of incorporating PEG onto the surfaces. The first is to copolymerize the material with PEG to form a homogeneous polymer. The second method is to take preformed particles containing functional groups such as carboxyl groups or amines and to activate the functional groups to facilitate reaction with the proper PEG derivative (e.g., an epoxy-terminated PEG) to produce a covalent bond. Without wishing to be bound by theory, it is expected that copolymerization would provide better assurance of complete surface coverage, but may require larger batches of starting reagent.

PEGs come in various forms. They can be linear or branched, of different molecular weights (chain lengths), and may be partially substituted (e.g., polyethylene glycol-polypropylene glycol co-block polymers, Polaxomers). The literature contains discrepancies as to the best choice of chain length and no one has studied the suitability of branched chains. One study, Gref et al., Science 263:1600-1603 (1994), concluded that polystyrene nanospheres with longer PEG chains, those greater than 10,000 Da, survived longest in the rat, but did not show direct evidence of surface coverage. Another study, Dunn et al., Pharma Research 11:1016-1022 (1994), concentrated on showing the importance of surface coverage density but did not compare these results with those results showing an effect as a function of PEG chain length. Importantly, long chain PEGs may sterically interfere with each other during the surface bonding procedure or during copolymerization. Thus, the surface may not be amenable to essentially complete surface coverage by long-chain PEGs, a level of coverage preferred to minimize the possibility of opsonization. Another study, Allen et al., Biochimica et Biophysica Acta 981:27-35 (1989), suggested that vascular survival is not enhanced by conjugating PEG chains longer than 5000 Da. It is known that the density of PEG coverage on the surface plays a role in vascular survival. To avoid surface recognition by small proteins, the spacing between PEG chains should be less than 5 nm. As the PEG surface density approaches this value, in vitro tests reveal that the sorption of proteins decreases steadily. Whereas higher molecular weight proteins are repelled at relatively low surface densities of PEG, lower MW proteins are repelled at relatively higher surface densities of PEG.

There are several biopolymers from which to choose in synthesizing the particles. Because of the amount of data and the proven biocompatibility, poly(lactic acid) (PLA) and poly(lactic-co-glycolic acid) (PLGA) polymers are preferred. Other applicable biopolymers include, but are not limited to, polyethylene glycol, dextran, albumin, starches and carbohydrates. Non-biodegradable polymers are also applicable and include polystyrene, silica-based particles, carbon or iron-carbon, gold, and titanium oxides, as these have demonstrated biocompatibilities.

Still other embodiments of the invention involve any medication, medicament, diagnostic substance or diagnostic agent known in the art being associated with a particle. Such compounds and compositions may be associated by being attached covalently or non-covalently to particles, or may be incorporated into the particle (e.g., co-polymerized with a surface coat of a particle), or encapsulated within a particle. Further, one or more molecules of a given compound or composition may be associated with a particle, or a mixture of compounds/compositions may be associated with a particle. In turn, certain applications will involve the administration of a homogeneous mixture of particles in terms of compounds/compositions associated therewith, or may involve heterogeneous mixtures.

In some embodiments, the invention is used to remove chemical or biological warfare agents from a soldier, a police agent or officer, and/or a noncombatant during an act of aggression, such as an act of terror or other crime of violence, a police action, or a war. An individual organism, such as a human who may be a member of a military force is exposed to an airborne deleterious substance such as a toxin after detonation of a biological weapon in an urban setting.

Included in a suitable military pack is a magnetic particle sequestration system. In response to the perceived threat of an airborne toxin, the military personnel removes an injectable device containing a magnetic particle bearing an antitoxin. In this example, a single antitoxin is bound to the magnetic particle surface. The invention contemplates a variety of other formats for antitoxin-particle attachment, such as a plurality of distinct antitoxins bound to a particle, a mixture or collection of particles collectively bearing a plurality of antitoxins, wherein each particle is attached to one type of antitoxin (a mixture of such particles refers to co-administration of the particles, whereas a collection of such particles does not require co-administration), and a plurality of particles, wherein each particle is attached to a plurality of distinct antitoxins. For example, an alternate embodiment provides a cocktail of particles where a single injectable device contains magnetic particles, each containing several different antitoxins (suitable for use where the actual toxin is unknown or is one of several possibilities). In yet other alternative embodiments, non-specific surface interaction between the magnetic particles and the blood-borne toxin are contemplated. Once injected, e.g., into the bloodstream, the magnetic particles flow through the blood and bind the specific toxin to which the individual was exposed. A complex between the toxin and the antitoxin bound to a magnetic particle then forms in the bloodstream.

After an appropriate circulation time, the individual, e.g., soldier, attaches a small filtration unit to an arm by inserting a dual lumen catheter into a vein. In an alternate embodiment, a dual access single lumen catheter may be used for the dual lumen catheter. The soldier waits until the magnetic particles have been completely removed before disconnecting the catheter from the arm. The time taken to remove a unit dosage of particles with a given filtration unit can be determined using no more than routine experimentation, and such information may be supplied with the unit as part of an instruction, e.g., in the form of a kit. The instruction may optionally include a margin for error to ensure that bound deleterious substances such as toxins are reliably removed in field situations, such as remote and/or hostile regions.

The extracorporeal magnetic filtration unit may consist of a feed tube that is branched into several smaller tubes or an array of smaller tubes that penetrate a magnetic field gradient contained in a housing. The magnetic field can be set up by a permanent magnet or an electromagnet that can be activated by, e.g., a small battery source. The tubes may pass under, over, or through the magnet to create a field gradient across the inner diameter of the tubes. The purified blood is then passed back into the human or animal through the feed tube, or through a separate tube(s).

A related aspect of the invention is drawn to a method of diagnosing a deleterious substance-induced condition. For example, a magnetic particle attached to a plurality of distinct antitoxins, optionally in combination with other particles of similar design but distinct antitoxin composition, are administered to an organism under binding conditions permissive for the binding of one or more deleterious substances to one or more administered particles (e.g., the normal in vivo conditions of a mammal), wherein the particle has an in vivo half-life of at least fifteen minutes, and preferably at least thirty minutes. Following administration, the particles are removed from the organism by exposing the particles to a magnetic field. The removed particles are then subjected to analysis to identify any bound deleterious substances. Any analytical technique known in the art is contemplated for use in identifying the deleterious substance(s), such as toxin(s), either with or without detachment of the deleterious substance(s) from the particle.

The following example provides a method to estimate target receptor site densities. Simulating a class A agent (Centers for Disease Control and Prevention classification) exposure, such as Bacillus anthracis exposure, by injecting 0.6 μg of lethal factor (LF) into a rat (300 g weight, 25 mL blood volume) leads to a LF blood concentration of 24 ng/mL or 7.5 pmol/rat (MW 80-90 kDa). Particles that have been used yield data indicating a surface receptor capacity of at least 1 μequiv/mg or 1 μmol/mg for univalent ligands. Thus, an anticipated injection of 10 mg of particles into a rat would have a capacity to bind 10 μmol of class A toxin, barring steric hindrance. This value is on the order of 10⁶-fold greater than the theoretical capacity needed to quantitatively bind LF toxin in the blood. The theoretical capacity was determined by assuming that the lethal factor dose leading to 24 ng/mL would occupy the 10 mg of nanoparticles uniformly. Thus the capacity would be (24 ng/mL)×(25 mL/10 mg)=60 ng LF per mg of particles. Appropriate multiplication by the molecular weight of LF yields the theoretical capacity in nmol per mg. Assuming a steric hindrance offered by an 80 to 90 kDa LF protein (15 nm diameter projected image) on a 400 nm magnetic nanoparticle (10 mg injection), a binding capacity of 0.7 nmol LF protein is expected, which is still a factor of 100 greater than necessary for quantitative LF removal from the blood.

MPs bind to blood-borne substances, such as toxins, in various ways. Examples include, but are not limited to, non-specific binding such as by van der Waals forces attracting the substance/toxin to be removed, i.e., due to mass and surface charge effects; and specific binding, such as ligand-receptor binding; chelation; antigen-antibody binding; chemical and/or steric binding; direct antitoxin-toxin binding, incorporation (the active or passive uptake into cells); engulfment (active ingestion of particles by a cell(s)); or effective inactivation or sequestration of the toxin without MP removal by direct or indirect toxin-antitoxin interaction, as well as any other form of binding known in the art.

Any technique known in the art for associating a specific binding partner (or medication or diagnostic agent) with a magnetic particle may be used. For example, antibodies or chelating ligands may be attached to an MP by attaching them directly to the particle surface using short-chain functional groups such as carboxyl and/or epoxy bridges, or by attaching them to the MP surface coating, e.g., PEG or PEG derivative chains extending from the particle surface. In considering the former procedure, it should be kept in mind that the receptor groups may be sterically hindered from encountering the toxins present in solution due to the long PEG chain neighbors. Also, because the receptors would be attached directly to the surface, they would be competing with the PEGs for surface coverage. This condition inherently limits the surface density of PEGs and facilitates opsonization and macrophage removal of the MPs in vivo. Therefore, attachment of the specific binding partner(s) to the surface coating of an MP is preferred. It has been demonstrated that the terminal groups of PEG chains can be activated and covalently bound to a variety of functional groups. Consistent with this observation, streptavidin has been attached to the terminal groups of PEG (300 and 2000 Da). The invention comprehends the generic attachment of a variety of medications or diagnostic substances to a magnetic particle (e.g., nanoparticle) substrate, as well as specific binding partners and inactivating agents useful in detoxification. Further, the invention comprehends incorporation (e.g., co-polymerization with a particle coating) as well as encapsulation of an active compound or composition within a particle that is preferably semi-permeable. For instance, by coupling or binding streptavidin to the terminal groups of the PEGs, one has a generic method of indirectly attaching biotinylated antibodies to the surface of a magnetic particle.

To facilitate proper function, antibodies are preferably conjugated to maximize surface density. Antibodies are large molecules (10 to 100 kDa), but their active regions can be quite small. Thus, antibody fragments can be used to increase the relative receptor site density (due to diminished steric effects) on the particle surface, while maintaining binding specificity and stability. Accordingly, an antibody fragment is a preferred binding partner for attachment to magnetic particles, resulting in higher binding capacities for toxins than is achievable using complete antibodies. Of course, the invention comprehends attachment of any known form of an antibody, including whole antibodies, single-chain antibodies, antibody fragments, chimeric antibodies, humanized antibodies, or any other form known in the art.

Another aspect of the proposed technology is indirect or generic attachment of a variety of anti-toxins to the magnetic particle (e.g., nanoparticle) substrate. For instance, by coupling or binding streptavidin to the terminal groups of the PEGs, one has a generic method of indirectly attaching biotinylated antibodies to the surface of a magnetic particle.

A wide variety of toxins or biological substances are removable from humans and animals using the methods of the invention. For example, toxin and biological substance candidates include, but are not limited to, medications, any toxins or substances entering the body via inhalation, injection, ingestion, transdermal or other topical application or iatrogenic exposures and implantations; chemical, biological, and radioactive/radiological toxins and biohazards of various chemical and physical composition; infective vehicles such as a prion; bacterial, fungal, and viral agents; and their respective toxins. A representative list of suitable toxins and biological substances is provided in Table I.

In one example, a single medication or diagnostic substance is bound to the magnetic sphere surface, incorporated into the sphere matrix, encapsulated in the sphere, or any combination thereof. The invention contemplates a variety of other formats for medication- or diagnostic substance-particle attachment, such as a plurality of distinct medications and/or diagnostics bound to a particle, a mixture or collection of particles collectively bearing a plurality of medication or diagnostic substances, wherein each particle is attached to one type of medication and/or diagnostic (a mixture of such particles refers to co-administration of the particles, whereas a collection of such particles does not require co-administration), and a plurality of particles, wherein each particle is attached to a plurality of distinct medications and/or diagnostics. For example, in some embodiments, a “cocktail” of particles is provided where a single injection of functionalized spheres contains magnetic particles, each containing several different medications and/or diagnostics. These embodiments are well-suited for emergency treatment situations, e.g., in field applications where there is insufficient information and time to determine a precise course of treatment, including medications or detoxification. In yet other alternative embodiments, non-specific surface interaction between the magnetic particles and the implanted stent or seed are contemplated. Once injected, e.g., into the bloodstream, the magnetic particles flow through the blood and bind to the magnetized stent or seed. Magnetic attraction of the medication or diagnostic substance which is coupled to the magnetic spheres and the implanted stent or seed magnetized by an external magnetic source will lead secondarily to either chemical binding of the medication or diagnostic substance at the stent or seed site or alternatively to simple deposition of a medication or diagnostic substance in the tissue or organ surrounding the stent/seed.

After an appropriate circulation time and exposure to the magnetized stent or seed, a time readily determinable by one of skill in the art using routine procedures, most or all of the functionalized magnetic spheres will have bound to the stent or seed and/or be deposited in the tissue and/or organ surrounding the stent and/or seed. At this time, magnetization of the stent or seed is no longer needed as most or all of the magnetic particles are sequestered from the bloodstream. However, the medication or diagnostic substance remains at the stent or seed site and/or the surrounding tissue/organ fixed by either a chemical binding or simple deposition.

Circulating functionalized spheres can subsequently be removed from the bloodstream using a novel technique called extracorporeal magnetic separation. This device consists of a short, small diameter, non-clotting loop circulating blood directly from an artery, or vein, back to a vein (artery-vein circuit or vein-vein circuit). In midsection the tube branches into several smaller tubes or an array of smaller tubes that penetrate a magnetic field gradient contained in a housing. The magnetic field traps and sequesters circulating magnetic spheres in a specific section of the tube. Blood free of magnetic spheres is then returned to the body by the tube. The sequestration unit can contain either a permanent magnet or an electromagnet activated by, e.g., a small battery source. The blood-circulating tubes may pass under, over, or through the magnet to create a field gradient across the inner diameter of the tubes.

Particle Size Distribution

Particles synthesized of polystyrene can be made into various sizes ranging <70 nm to >10 mm. These substrates are already available from various manufacturers in the U.S. and elsewhere. Biodegradable particles, e.g., spheres, made from poly(lactic acid) (PLA) or poly(lactic-co-glycolic acid) (PLGA) can be made in various size ranges but are generally more polydisperse than the polystyrene particles.

To make PLA particles of a size less than 200 nm mean diameter, the synthesis protocol of Gref, et al., Science 263:1600-1603, 1994, was largely followed. The lactic acid:glycolic acid ratio was 3:1 in each case and PEG was 10% by weight (PEG MW varied from 5 to 20 kDa). The copolymer was dissolved in solvent (ethyl acetate or methylene chloride, 25 mg/2 mL), poured into 30 mL of deionized water, and an oil-in-water emulsion was formed by vortexing (30 seconds) and sonicating (1 minutes, 40 W output). The organic solvent was slowly removed by evaporation and gentle stirring at room temperature for 2 hours. The spherical particles were monomodal in size at 140 nm diameter.

Another method for particle synthesis is based on the disclosure of Dunn et al., J. Controlled Release, 44:65-76 (1997). Dunn et al. taught nanoparticle synthesis using PLGA (75:25, 63 kDa, ResomerRG755) polaxamer407 (11,500 MW), poloxamine904 (6700 MW), and poloxamine908 (25,000 MW). The nanospheres were prepared using an oil-in-water technique. The PLGA was prepared in the presence of surfactant and also without surfactant. The nanoparticles prepared without surfactant were subsequently incubated in surfactant solution. PLGA was dissolved in 5 mL of acetone to make a 0.5% or a 0.25% (w/v) solution. This solution was added dropwise into a water solution containing or not containing the surfactant (the poloxamer/ines listed above) while mixing at ambient temperature. The resultant material was passed through a 1 μm filter after the organic solvent had evaporated. The nanoparticle size was 99-122 nm depending on the surfactant copolymer used, with a very small standard deviation (<1 nm). Any of the methods for particle synthesis may also be combined with membrane emulsification to obtain monodisperse magnetic particles.

Larger biodegradable microspheres can be synthesized using water-in-oil-in-water techniques, see O'Hagan et al., Immunology, 73:239-242 (1991). O'Hagan et al. disclosed that PLGA (ResomerRG503, 9 kDa 50:50) was dissolved in dichloromethane to 6% (w/v) and this solution was homogenized with 6% antigen (ovalbumin) in water. This emulsion was added to a much larger volume of 5% PVA solution and homogenized. The typical antigen concentration was 1% (w/v) and the size was 5.34 μm, as determined by laser diffractometer measurements.

Bimodal Particles

A particle can be synthesized to include several functional groups to sequester a number of substances, such as medications, diagnostics or toxins, thereby producing bi- or multi-modal particles For instance, if one were exposed to several radionuclides typically co-produced as fission products, it would be desirable to concurrently remove the chemically dissimilar radionuclides, e.g., radioactive isotopes of cesium and cobalt, so produced. Suitable for such a treatment is a magnetic particle that is synthesized and embedded with crystalline silicotitanate, having phosphinic acid groups attached to the magnetic particle surface, thus allowing simultaneous removal of cesium and cobalt ions. A similar approach is followed for removal of several antigens or for the delivery of several medicaments and/or diagnostic agents. The particle can be synthesized to accept or attach several antibodies.

Magnetic Component

The magnetic character of the particles is established by encapsulating or precipitating iron oxides or other magnetic material inside and/or on the polymeric matrix of particles. To encapsulate material, the magnetic crystals are introduced into the organic phase with, e.g., PLA, PLGA, or styrene, during synthesis. Typically 1-20 wt % of magnetic crystals is necessary to achieve appreciable magnetic moment. There are several magnetic materials contemplated, including permanently magnetic materials and paramagnetic materials. Magnetite (Fe₃O₄) and maghemite (g-Fe₂O₃) are most common, being commercially available or easily precipitated in the laboratory. Other magnetic materials include metallic iron, cobalt, or nickel, but steps must be taken to protect (passivate) the surfaces from oxidation. Permalloy, cobalt ferrite (CoFe₂O₄) and rare earth-based magnetic materials (e.g., NdFeB, SmFe₂, TbFe₂, TbDyFe, NdCo₅, SmCo₅, LaCo₅, CeCo₅, or PrCo₅) are also available.

In situ precipitation is accomplished by the same method used to produce magnetite or maghemite nanocrystals in solution except that the ferric and ferrous iron salts are introduced into the solution following nano/microparticle synthesis and allowed to equilibrate with the particles. Caustic (e.g., NaOH) is used to increase pH and cause iron oxide to precipitate from solution. Iron oxides will also precipitate within and on the surface of the particles.

Removal of the Magnetic Particles

A preferred magnetic separator unit utilizes small permanent or electromagnets attached to the body of a specialized closed-loop catheter system. Common to all preferred design options, the blood is diverted from the body through flow tubes with a diameter ranging from about 100 nm to about 10 mm. These tubes are immersed in a magnetic field gradient causing the magnetic particles to deflect towards, and collect at, the tube wall. The precise geometry of the tubing system (size, material, coating, length, shape, and the like) is defined based on the particular application (i.e., in-field versus unit based) and user level of training (i.e., self- versus helper-applied). Some applications will require rapid removal of particles and thus high flow rates through the magnetic separator unit. Such flow rates preferably will be achieved by designing tube arrays (a plurality of tubes) that pass through the magnetic field gradient. In some instances, large magnetic field gradients may be necessary and used. In such scenarios, fewer tubes of a large diameter will be more appropriate to achieve rapid separation of particles form the blood.

In a relatively straightforward embodiment, the method of extracorporeal composition sequestration (i.e., toxin removal) comprises:

1) A short biocompatible tubing (heparinized or otherwise treated to minimize side effects, e.g., clotting of blood within the tubing system).

2) An indwelling catheter connected to each tubing end, with both catheter ends inserted into at least one blood vessel. In a preferred embodiment, one of the catheters is inserted into a human artery, providing inflow to the tubing, and the other catheter is inserted into a vein of that human, providing return blood flow to the body. Alternatively, both tubing ends are connected to a dual lumen catheter, which then is inserted either into a human artery or vein. Connected in such ways, the tubing/catheter system becomes a closed-loop module for extracorporeal blood circulation.

3) An external magnetic field adjacent to a circumscribed segment of the extracorporeal tubing. The magnetic field strength is calculated to sufficiently trap magnetic particles within the tubing and against the flowing bloodstream. To retain larger amounts of magnetic particles, the tubing can alternatively contain a small extension or pouch in the area of magnetic field exposure in order to support accumulation of magnetically trapped particles over time.

4) Alternatively, the tubing system used for extracorporeal blood circulation can itself be temporarily or permanently magnetized (as opposed to a separated magnet unit adjacent to the tubing). Removal of magnetic particles with bound toxin(s) from the bloodstream is then achieved by sequestration of the particles to an inner tubing wall.

Additional embodiments of the magnetic separation unit include the incorporation of sensors to detect medication, diagnostic substance, or toxin during magnetic dialysis or additional chambers to divert magnetic particles for assay or analysis. In this manner, the technology would provide an immediate analyte for assay (e.g., forensics). As an example, the separation unit is outfitted with appropriate photosensors attached to a translucent window of the separation chamber. A separate accessible chamber contains a cocktail of fluorescent agents or a single fluorescent agent. Exposure of the magnetic particles trapped in the chamber with the fluorescent markers will cause co-conjugation of the fluorescent markers to the toxin (i.e., the toxin, and in other embodiments the medication or diagnostic agent, is bound to the particle surface and to the fluorescent agent). A wash solution, contained in a separate accessible chamber, is brought into contact with the particles to remove unbound fluorescent markers. Photosensors inside the unit then detect fluorescence of defined wavelength(s) to determine the type and amount of medication, diagnostic substance, or toxin bound to the particles. Because the magnetic particles have concentrated the medication, diagnostic substance, or toxin onto the surface, a concentrated analyte is provided to increase detection sensitivity. The unit can be equipped with readouts to facilitate medication, diagnostic substance, and/or toxin identification. Alternatively, the fluorescent markers are pre-conjugated to the medication or diagnostic substance, or to an anti-toxin, on the particles, with the markers exhibiting a wavelength shift once conjugated to the medication, diagnostic substance, or toxin. The photosensors would detect the type and concentration of bound material, as described above, using the attached photosensors. This approach can be extended to include other analytical methods not dependent on the detection of fluorescence, as would be known in the art. Additionally, magnetic sensors can be incorporated inside the unit to monitor the extent of magnetic particle removal.

The magnetic separator separates the particles from the biological fluid(s), such as blood, which is typically accomplished by providing a sufficient magnetic field to draw the particles to the wall and hold them there under shear flow. Appropriate magnetic fields are 100-100,000 Gauss and field gradients of 0.1-2000 T²/m. A total cleansing of a typical human body will require a flow rate of about 1-200 ml/minute. Lower flow rates can easily be obtained from a large bore venous puncture at mid-arm level. A simple siphon hand-pump will ensure proper flow rate. Higher flow rates are achieved with a commonly performed femoral arterial puncture with a double bore needle (double lumen [inflow and outflow] catheterization avoids a second vascular puncture). The techniques involved are known to those of skill in the art.

Preferably, anticoagulation treatment is confined locally in the perfusion chamber by dissolution of heparin from the walls of the tubes into the flowing fluid, thereby permitting adequate anticoagulation locally without introducing it systemically. Shear rates and stresses are kept at levels compatible with relatively minimal clot formation and thrombosis in the design of the magnetic field, so that particle removal or “filtration” is quantitative and highly efficient. Preferably, the length of the device and its size and weight are minimized. The device preferably includes a clot filter at the blood return port.

The described technology is applicable to humans, other animals, and plants.

Magnetizable Stents and Seeds

Stents and seeds according to the invention exhibit the defining characteristics of being magnetizable and being biocompatible; as used. Although the stent or seed per se may not be biocompatible, in use these devices are rendered biocompatible, e.g., by applying a biocompatible surface coating, e.g., PEG or PLGA. The stents and seeds are also capable of magnetically attracting the magnetic particles described herein. Preferably, the stents and seeds are capable of attracting and retaining the magnetic particles. Any known composition, form, or number of materials is contemplated for use in these stents and seeds, provided that the defining characteristics of magnetizabililty and biocompatibility are retained. Of course, preferred stents also exhibit the capacity to maintain the patency of a lumen in which they are found or used. The stents and seeds may be a continuous, or intermittent, solid material, such as a metal, a metal alloy, a metal-impregnated plastic, and the like. As disclosed herein, these stents and seeds are useful in facilitating the targeted delivery of medications, diagnostic agents, binding partners for deleterious substances (e.g., toxins), and inhibitors/inactivators of deleterious substances. The stents and seeds are capable of facilitating the targeted delivery of these compounds and compositions directly, provided that such compounds or compositions exhibit a magnetic moment. More typically, however, the stents and seeds facilitate the targeted delivery of these compounds and compositions indirectly, as a result of the association of such compounds and/or compositions with magnetic particles, as described above.

Suitable Medications or Diagnostic Substances

Conventional medications or diagnostic substances (e.g., antibodies, ligands, chelators) can be chemically attached to the surface or terminal groups of the particle surface coating, e.g., a PEG. Specifically with the use of antibodies, any steric hindrance between an antibody and PEG chains may be addressed by using a single-chain antibody or an antibody fragment, including chimeras and humanized versions of such sc antibodies and fragments. These fragments, and sc antibodies, include the antigen binding component of at least one chain of an antibody. In alternative embodiments, the compound or composition is any medication, diagnostic agent, toxin binding partner, toxin inactivator, or toxin sequestering agent known in the art.

Medical Applications

This technology will be integrated into daily clinical practice for human drug delivery and treatment. Many medical applications are contemplated, from the simplest form, such as treatment of acute stent occlusions, i.e., as in acute coronary artery disease, which could be treated by this technology with clot buster plasminogen activator-loaded magnetic particles attached to the magnetized stent, e.g., a coronary stent, leading to successful re-opening of the stent lumen (thrombolysis) or to more complex drug delivery into the tissue or organ surrounding a stent or seed, such as in a patient with localized cancer being treated with irradiating medications in order to diminish tumor burden. The invention is useful in acute and/or chronic treatments. Chronic body treatment is possible with the present method for at least the following reason: repeated injections of drug-loaded magnetic particles can be delivered to the target stent or seed site, achieving chronic treatment while using a single drug-loaded magnetic carrier batch or multiple differently loaded functionalized particles. Such an option will provide a relatively non-invasive, yet targeted, therapy conveniently performed in the ambulatory hospital or home setting. Therefore, treating chronic diseases, disorders, or conditions with repeated or continuous injection sessions is contemplated and clinically useful.

Many other illnesses are amenable to this form of treatment, and placement of the magnetizable or magnetic stent is not limited to placement in a blood vessel of suitable size, or even to a vessel per se. In many treatment methods, for example, the magnetizable or magnetic stent is placed directly into the tissue, organ or organ system, e.g., a tumor tissue, such as by surgery (e.g., cancer surgery involving abdominal or pelvic tumor removal operations) or by stereotactic implantation of an otherwise surgically inaccessible tumor bed such as diffuse brain tumors.

The medication- or diagnostic agent-containing MPs can be delivered using any known route of administration, including any suitable form of injection, transdermal delivery, intraluminal delivery (e.g., intraintestinal delivery by injection or spray), topical administration and by inhalation. By way of example, the delivery of medication- or diagnostic agent-containing MPs by inhalation would involve initial entry of the MPs into the respiratory tract, through assisted or unassisted respiration. Subsequently, the MPs would enter the circulatory system via the pulmonary endothelium and eventually arrive at the site of the magnetizable stent or seed, where they would be retained for some period of time. This route of administration is expected to be convenient and to provide for the rapid uptake of MPs. MPs of relatively small size (mean diameters in the low nanometers) are expected to directly reach the terminal regions of the respiratory tract, i.e., the alveoli, where uptake by capillaries is expected. Larger MPs (mean diameters in the low micrometer range) are also contemplated as usefully administered by inhalation, in that some of these larger MPs are expected to evade bronchial clearance mechanisms, contact the hydrophilic alveolar surface, degrade into smaller medication- or diagnostic agent-containing particles for transalveolar uptake into the bloodstream, and eventually become sequestered at the stent or seed site.

Suitable Antitoxins

Conventional anti-toxins (e.g., antibodies, ligands, chelators) can be chemically attached to the surface or terminal groups of the particle surface coating, e.g., a PEG. Specifically with the use of antibodies, any steric hindrance between an antibody and PEG chains may be addressed by using an antibody fragment. These fragments include the biologically active component of the antibody. Currently there are many antibodies available, depending on the antigen, and some of these antibodies are listed in Table I.

Table I includes toxins of both direct biological threat relevance (bold) as well as toxins of medical importance. Columns 1-3 identify the toxin, source organism and protein characteristics where relevant, respectively. The column “gi” contains a representative “gene identification” number of an amino acid sequence of protein toxins; “#aa” provides the number of amino acids; “PDB” provides a protein data base identifier of the atomic coordinates of a representative structure (where available); “Ab vendor” indicates a commercial supplier of antibodies to the toxin (vendor acronym: catalog number; see Table II for vendor identification). In the gi, #aa, and PDB columns, “NP” indicates that the toxin is not a protein; “-” indicates information has not been found. In the PDB column, structures of homologs are indicated by “h” preceding the file identifier; partial structures are indicated by “p”.

TABLE I
Toxin Summary

| Toxin | Organism | Protein | gi | #aa | PDB | Ab vendor |
|---|---|---|---|---|---|---|
| Abrin | Abrus precatorius | RIP | 999849 | 251 | 1ABR | |
| | | | 999850 | 267 | | |
| Adenylate cyclase | Bordatella | Hemolysin; pore-former | 580668 | 1706 | h1K8T | BBI: T-4464.0400 |
| | pertussis | | | | | BCG: 7394-9009 |
| Aerolysin | Aeromonas | Pore-former | 19550929 | 380 | 1PRE | |
| | hydrophila | | | | | |
| Aflatoxins | | | NP | NP | NP | |
| Alpha toxin | Staphylococcus | Pore-former | 2126575 | 319 | 7AHL | BCG: 8400-9009 |
| | aureus | | | | | |
| Androctonin | Androctonus | Antimicrobial peptide from | 6980632 | 25 | 1CZ6 | |
| | Australia | scorpion | | | | |
| Anthrax toxin | Bacillus anthracis | Edema factor (EF) | 16031479 | 779 | 1K8T | |
| | | [Adenylate cyclase] | | | | |
| | | Lethal factor (LF) | 21392848 | 809 | IJ7N | |
| | | (protease) | | | | |
| | | Protective Antigen (PA) | 10880948 | 764 | 1ACC | |
| | | | | | | USB: B0003-05 |
| Botulinum toxin | Clostridium | Protease; neurotoxin | 16580759 | 1291 | | BCG: 2119-3100 |
| (A-G) | botulinum | | | | | BCG: 2119-3100 |
| | | | | | | BCG: 2119-2990 |
| | | | | | | CBI: CR7002R |
| C2 toxin | Clostridium | ADP-ribosyltransferase | 3183651 | 431 | h1QS1 | |
| | boltulinum | | 3478672 | 721 | h1ACC | |
| C3 toxin | Clostridium | ADP-ribosyltransferase | — | — | — | |
| | botulinum | | | | | |
| Cholera enterotoxin | Vibrio cholerae | ADP ribosylase - activates | | | | ACS: YCC-340-601 |
| (ctx) | | Adenylate cyclase | | | | BCG: 9540-1559 |
| | | | | | | BCG: 9540-0008 |
| CLDT | Escherichia coli | G2 block | — | — | — | |
| CNF | E. coli | Deamidase | — | — | — | |
| Conotoxin-alpha | Various snails | Nicotinic ligand-gated ion | 12084187 | 13 | 1E76 | |
| Conotoxin-alpha-A | (cone shells) | channel blocker | | | 1P1P | |
| Conotoxin-psi | | | | | | |
| Conotoxin-omega | | Voltage-gated Ca channel | | | 1DW4 | |
| | | blocker | | | | |
| Conotoxin-mu | | Voltage-gated Na channel | | | 1GIB | |
| Conotoxin-delta | | blocker | | | | |
| Conotoxin-kappa | | Voltage-gated K channel | | | 1AV3 | |
| | | blocker | | | | |
| Cytotoxic | E. coli | Rho activator | 14719449 | 295 | 1HQ0 | |
| necrotizing factor, | | | | | | |
| type 1 | | | | | | |
| Deoxynivalenol | | | | | | |
| Dermonecrotic toxin | Bordatella | Deamidase | 2120991 | 1451 | hp1HQ0 | |
| | pertussis | | | | | |
| Diacetoxyscirpenol | | | | | | |
| Diptheria toxin (dtx) | Corynebacterium | ADP ribosylase of | | | | MBSI: MAB768P |
| | diphtheriae | elongation factor 2 | | | | MBSI: 769P |
| EAST | E. coli | ST-like (expected) | | | | |
| Epsilon toxin | Clostridium | | 282478 | 328 | h1PRE | |
| | perfringens | | | | | |
| Equinatoxin II | | | | | 1LAZ | |
| Erythrogenic toxin | Streptococcus | Like TSST | 1877430 | 251 | 1B1Z | |
| | pyogenes | | | | | |
| Exfoliatin toxin | Staphylococcus | Cleavage of epidermal cells | | | | |
| | aureus | (superantigen) | | | | |
| Exotoxin A | Pseudomonas | Similar to diphtheria toxin | | | | ACS: BYA-2112-1 |
| | aeruginosa | | | | | LBLI: 760 |
| Flavocetin | | Snake venom anticoagulant | | | | |
| Hemolysin | E. coli | Pore-former | | | | |
| Huwentoxin-I | Ornithoctonus | Neurotoxin; spider | 6136076 | 33 | 1QK6 | |
| | buwena | | | | | |
| Huwentoxin-II | Ornithoctonus | Neurotoxin; spider | 13959612 | 37 | 1I25 | |
| | buwena | | | | | |
| Huwentoxin-IV | Ornithoctonus | Neurotoxin; spider | 21542276 | 35 | h1QK6 | |
| | buwena | | | | | |
| Iota toxin | Clostridium | ADP-ribosyltransferase | 2127361 | 454 | h1QS1 | |
| | perfringens | | 2127362 | 875 | h1ACC | |
| Leukocydin F | Staphylococcus | | 5822485 | 299 | 2LKF | |
| | aureus | | | | | |
| Listeriorlysin O | Listeria | Pore-former | | | | |
| | monocytogenes | | | | | |
| LT toxin | E. coli | Similar to cholera toxin; | | | | |
| | | heat labile toxin | | | | |
| Mastoparan | Bee (hornet, wasp) | Cytoactive peptide; induces mast | 14719630 | 15 | 1D7N | |
| | venom | cell degranulation and the release | | | | |
| | | of histamine. | | | | |
| Nivalenol | | | | | | |
| Nodularin | Nodularia | Inhibitor of Ser/Thr-specific | 5821779 | 4 | 1AY3 | |
| | spumigena | protein phosphatases | | | | |
| Perfringolysin O | Clostridium | Pore-former | 3401988 | 500 | 1PFO | |
| | perfringens | | | | | |
| Perfringens | Clostridium | Stimulates adenylate cyclase | | | | |
| enterotoxin | perfringens | | | | | |
| Pertussis toxin (ptx) | Bordatella | ADP ribosylase - blocks | | | | |
| | pertussis | inhibition of Adenylate | | | | |
| | | cyclase | | | | |
| Pneumolysin | Streptococcus | Pore-former | | | | |
| | pneumoniae | | | | | |
| Pyrogenic exotoxins | Staphylococcus | Superantigen | | | | |
| | pyogenes | | | | | |
| Ricin | Ricinus communis | Ribosome inhibitor protein | 18655838 | 267 | 1IL9 | |
| | | (RIP) | | | | |
| Saxitosin | Shellfish | Na-channel blocker | NP | NP | NP | |
| Scorpion toxin | Centruroides | Neurotoxin | 20150615 | 66 | 1JZA | |
| | sculpturatus | | | | | |
| Shiga | Shigella | subunit 1A | 21636533 | 365 | 1DMO | |
| | dysenteriae | rRNAse | | | | |
| | E. coli | subunit 1B | 21636534 | 89 | | |
| | | subunit 2A | 21636563 | 319 | | |
| | | subunit 2B | 21636537 | 89 | | |
| ST toxin | E. coli | Heat stable toxin | | | | |
| Staphylococcus | Staphylococcus | Superantigen | | | | |
| enterotoxins | aureus | | | | | |
| Streptolysin O | Streptococcus | Pore-former | 19745333 | 574 | h1PFO | |
| | pyogenes | | | | | |
| T-2 toxin | | | | | | |
| Tetanus toxin | Clostridium tetani | Protease | | | | |
| Tetradotoxin | Fugu | Na-channel blocker | NP | NP | NP | |
| (TTX) | | | | | | |
| Toxic shock | Staphylococcus | Superantigen | | | | |
| syndrome toxin | aureus | | | | | |
| (TSST-1) | | | | | | |
| Toxin A | Clostridium | Glucosyltransferase | 98593 | 2710 | hp1HCX | |
| | difficile | | | | | |
| Toxin B | Clostridium | Glucosyltransferase | 98597 | 2366 | hp1HCX | |
| | difficile | | | | | |

Related Molecules

| | | | | | |
|---|---|---|---|---|---|
| Anthrax toxin | Homo sapiens | Tumor endothelial marker 8; | 16933553 | 333 | h1JLM |
| receptor | | | | | |

TABLE II
Vendors

| Abb. | Company Name | City | Phones | Fax |
|---|---|---|---|---|
| ACS | Accurate Chemical and | Westbury, NY 11590 | (516) 333-2221 | (516) 997-4948 |
| | Scientific | | (800) 645-6264 | |
| BBI | Bachem Bioscience Inc. | King of Prussia, PA | (610) 239-0300 | (610) 239-0800 |
| | | 19406 | (800) 634-3183 | |
| BCG | BIOTREND Chemikalien | Cologne, D-50933, | 49-221- | 49-221- |
| | Gmbh | DE | 9498320 | 1949832 |
| CBI | Cortex Biochem Inc. | San Leandro, CA | (510) 568-2228 | (510) 568-2467 |
| | | 94577 | (800) 888-7713 | |
| LBLI | List Biological Laboratories | Campbell, CA 95008 | (408) 866-6363 | (408) 866-6364 |
| | Inc¹ | | (800) 726-3213 | |
| MBSI | Maine Biotechnology Services | Portland, ME 04103 | (207) 797-5454 | (207) 797-5595 |
| | Inc. | | (800) 925-9476 | |
| USB | US Biological | Swampscott, MA | (800) 520-3011 | (781) 639-1768 |
| | | 01907 | | |

For the sequestration of radionuclide(s) or hazardous metal(s) from the blood, ligands or chelators have been developed for in vitro and in vivo applications. Two prime examples include the use of Prussian Blue (hexacyanoferrate) for transition metals and cesium and DTPA for trivalent metals, lanthanide, and actinide elements. These ligands can be chemically attached to the surface of the particles, embedded in the surface of the particles as nanocrystals, or conjugated to terminal PEG groups. Because the targets are atoms and not macromolecules (as in the antigen case), the atoms should be able to freely cross the PEG barrier to the particles and penetrate the surface to reach ligands bound there. However, other ligands, such as phosphinic acids, phosphine oxides, calixarenes, EDTA, and crystalline silicotitanates, have demonstrated affinity for certain radionuclides in the lanthanide and actinide series, as well as for cesium, cobalt, and strontium and such ligands are expected to be useful in in vivo applications. Unlike systemic injection of free chelators, the binding of chelators to the nanoparticle surface, possibly protected from an immune response by the PEG surface coating, can be injected into the body because they will be recovered following treatment, thus avoiding toxicity issues.

Medical Applications of Detoxification Aspects of the Invention

This technology will be integrated into daily clinical practice for human detoxification treatment. Many medical applications are contemplated, from the simplest form such as in emergent detoxification of patients with acute and subacute drug, medication, and toxin overdose/intoxication syndromes to much more complicated schedules such as intermittent, toxin dose-adjusted, chronic detoxification treatment to slowly detoxify body toxin stores in tissue other than the blood as well as toxin stores in organs and organ systems. The invention is also useful in the acute and/or chronic removal of toxin precursors. Chronic body detoxification of toxins stored in organs is possible with the present method for at least the following reason: toxins from the brain, liver, bone, or any other organ are in physiological equilibrium with the blood. Removing toxins chronically (either intermittently or continuously) from the blood—even when blood stores for a particular toxin are only minute—will ultimately lead to detoxification of the organ as organ-deposited toxin will re-establish equilibrium concentrations in the organ and in the blood, with a net flow of toxin from the organ(s) to the blood, where the toxin is removed, sequestered, or otherwise effectively inactivated. Therefore, treating chronic diseases, disorders, or conditions with repeated or continuous detoxification sessions is contemplated and clinically useful. As described above, the toxins contemplated by the invention include exogenous and endogenous toxins. An example of the latter would be an anti-self antibody, or autobody characteristic of some autoimmune disorders such as rheumatoid arthritis. The diseases, disorders, and conditions amenable to treatment with these methods include, but are not limited to, autoimmune, hematological, and rheumatological diseases; tumor treatment, including removal of tumor treatment agents/drugs from the blood; neurological, cardiovascular, respiratory, dermatological and other autoimmune and inflammatory diseases; removal of endogenous and exogenous toxins; infectious and other non-inflammatory or inflammatory diseases; and other diseases, disorders, and conditions known in the art to involve an endogenous or exogenous deleterious biological substance or composition, i.e., a toxin.

As in the medication- and diagnostic agent delivery aspects of the invention, the removal of deleterious substances can involve administration of MPs containing specific binding partners, or specific deleterious substance deactivating (e.g., toxin deactivating) compounds by any route known in the art, including any suitable form of injection, transdermal delivery, intraluminal delivery (e.g., intraintestinal delivery by injection or spray), topical administration and by inhalation. As in the other aspects of the invention, inhalation would involve initial entry of the MPs into the respiratory tract, through assisted or unassisted respiration. Subsequently, the MPs would be mobile within the body, facilitating interaction with a deleterious substance. Eventually, MPs, with or without bound deleterious substance, would be removed from the body, typically involving the use of an external magnetic field generator. MPs without bound deleterious substance are contemplated as useful if they are capable of inhibiting or inactivating a deleterious substance in vivo. Alternatively, the MPs are sequestered for a period sufficient to allow the deleterious substance to be inactivated by magnetic interaction of an MP containing the deleterious substance with a magnetizable stent or seed. Inhalation of small MPs (mean diameters in the low nm range) is expected to directly enter the alveolar microenvironment and enter alveolar capillaries. Larger MPs (mean diameters in the low micrometer range) are expected to evade bronchial clearance methods, in part, and those MPs reaching the hydrophilic environment of the alveolar surface, be degraded, and portions of such MPs, including MP portions attached to a specific binding partner or inhibitor/inactivator of a deleterious substance, are expected to enter the circulatory system via the alveolar capillaries, eventually becoming sequestered at a stent or seed site.

The following examples present preferred embodiments and techniques, but are not intended to be limiting. Those of skill in the art will, in view of the present disclosure, appreciate that many changes can be made in the specific materials and methods which are disclosed and still obtain a like or similar result without departing from the spirit and scope of the invention. In the following examples, Example 1 provides the materials and methods for sequestering a model toxin from fluids; Example 2 describes in vivo studies of toxin removal using the methods of the present invention; Example 3 describes an in vitro experiment involving capture of magnetic particles by a magnetizable coiled stent; Example 4 describes in vivo use of a magnetic particle associated with tissue plasminogen activator being used in conjunction with a magnetizable stent or seed to treat coronary artery disease; and Example 5 describes the use of a magnetic particle associated with methotrexate used with a magnetizable seed to treat a brain tumor.

### EXAMPLE 1

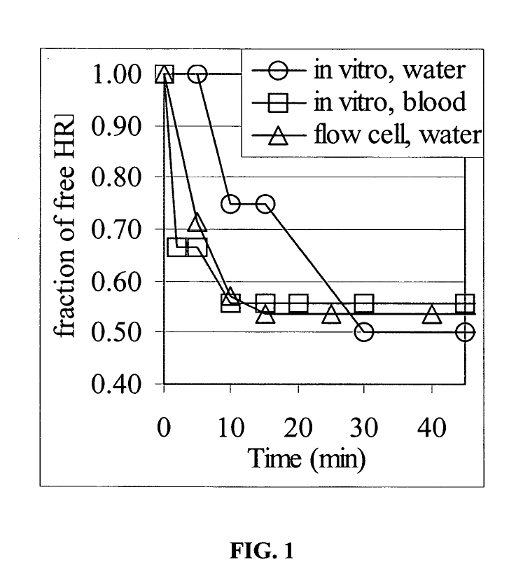

In vitro sequestration of a biotinylated enzyme from simple fluids and whole rat blood was performed under static and dynamic flow conditions. Particles were composed of nanocrystalline magnetite encapsulated in monodisperse polystyrene nanospheres. Several variations were tested, including various PEG length (MW 330-6000) and particle size (250-3000 nm). Streptavidin, the model receptor, was either bonded to the carboxylated terminal group of the PEG or attached directly to the nanoparticle surface. Biotinylated horseradish peroxidase (HRP) was used as the model “toxin.” The results shown in FIG. 1 indicate a reduction of the free enzyme to about 50% maximum levels in the blood in all tests. Equilibrium was reached within 20-30 minutes. The experiment demonstrates the operability of the methods and systems for deleterious substance removal/sequestration in vitro. Additional investigation, described e.g., in Example 2, demonstrates the operability of the invention in an in vivo environmental model of mammalian physiology.

### EXAMPLE 2

In vivo experiments, performed on retired breeder rats, included a) the design of a closed loop, adjustable flow, blood re-circulation unit permitting blood turn over and sampling over several hours in the live animal; b) kinetic studies of several candidate magnetic nanoparticles and toxins; and c) magnetic filtration experiments. In the latter investigations, continuous extracorporeal blood circulation was achieved via carotid-jugular cannulation and external pump support with filtration of magnetic nanoparticles using 1-mm diameter closed-loop tubing and a single NdFeB magnet (0.4 T at surface, 18 mm diameter). Experimental results on toxin sequestration from the rat are consistent with the in vitro data. In this experiment, a rat was injected with 15 μg HRP. After 5 minutes, 10 mg of streptavidin-coated magnetic particles (magnetite-embedded polystyrene, 400 nm) conjugated with PEG 2000 were injected. The results show a 50% reduction of HRP in the rat serum after 20 minutes of magnetic particle circulation, in agreement with the in vitro data.

### EXAMPLE 3

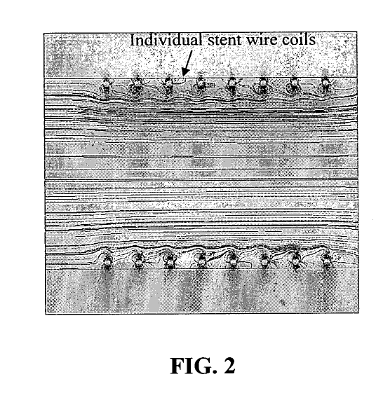

A stent of paramagnetic metal comprising eight coils, internal radius 2.5 mm, was prepared and placed into a plastic tube submerged in decalin, thereby matching the index of refraction so that a clear image can be captured. A permanent NdFeB magnet was placed against the tube adjacent to the wire coils. Non-porous magnetic particles containing 80% magnetite (w/w), 2.0 mm radius, were suspended in a fluid that flowed through the stent at 0.8 ms⁻¹. The NdFeB magnet-induced a magnetic field of 1.0 T perpendicular to the fluid flow and the long axis of the stent, resulting in capture of the magnetic particles, as illustrated in FIG. 2. Under our direction, the University of South Carolina conducted analyses of the results of this experiment, using Femlab, a commercially available magnetic field-fluid flow model. The magnetic particle capture efficiency was 26%.

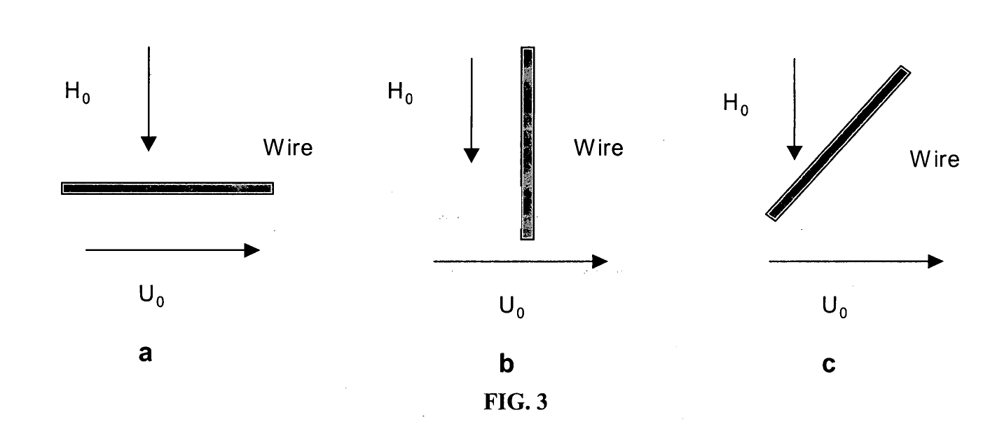

To better understand the capture of magnetic particles by a magnetizable stent, a computational model was developed and parameters were varied to determine the relationship between capture sensitivity and capture cross section (y-axis, distance between particle and stent where capture is possible): The stent can be modeled as a net made of metal wires. Thus, a piece of wire can be studied instead of the entire coil to simplify analysis. In practice, the external magnetic field H₀ can easily be kept perpendicular to the blood flow U₀. Thus, the position between H₀, U₀ and the wire, or magnetizable stent, could be schematically illustrated as shown in FIG. 3.

In FIG. 3a, the wire is parallel to the blood flow direction and the external magnetic field H₀ is perpendicular to both; in FIG. 3b, the wire is perpendicular to the blood flow direction, and the external magnetic field H₀ is parallel to wire while it is perpendicular to the blood flow; in FIG. 3c, there is an angle between H₀ and wire, and between U₀ and wire, however, H₀ is still perpendicular to the blood flow. Maximum force will be achieved when α equals 90°, i.e., the external magnetic field H₀ is oriented perpendicular to the metal wire or magnetizable stent, as demonstrated in FIG. 3a.

The magnetic force that acts on a magnetic particle is governed by

$$Fm = \frac{4 {πμ}_{0} {b}^{3} {a}^{2} {κ}_{p} {M}_{s} {H}_{0}}{3 {r}_{s}^{3}} {sin}^{2} α .$$

Based on the description above, the coil stent is modeled and shown in FIG. 4, where R is the radius of the expanded stent, and y₁ and y₂ are the distance from the center of stent coil wire to the center of particle 1 and particle 2, respectively (FIG. 4, top). A particle approaches a magnetizable wire in a magnetic field H₀ that is perpendicular to the blood flow U₀. Whether or not the particle will be captured by the magnetized wire in the magnetic field depends on capture cross-section, y_{c,} which is the maximum perpendicular distance that a particle could traverse from the center of the wire and still be captured by the magnetized wire (FIG. 4 bottom). For example, for particle 1, which is at the center of the coil stent, if the capture cross-section y_{c1} is larger than the distance between particle 1 and the center of stent coil wire y₁, then particle 1 could be captured by the system and attached to the coil stent wire; otherwise, particle 1 will pass through the stent along with the blood flow. The same thing would happen to particle 2: if yc₂>y₂, particle 2 could be captured. Hence, capture cross-section could help us predict the efficacy of this intravascular magnetizable stent based system: determining whether a magnetic particle will be captured by a magnetized stent in a predefined system.

The dimensionless capture cross-section is defined as follows (Ebner and Ritter, 2001):

$$λ = \frac{{y}_{c}}{{R}_{c}}$$

where y_{c} is the capture cross-section. R_{c} is the radius of the wire. If λ is much larger than 1, then we may get a desired capture cross-section and the method is feasible. However, if λ is smaller that 1, this delivery method may not be as good as expected.

In the graphic results shown in FIG. 5, the effect of individual particle size (a), type of magnetic phases and their percent composition in the particle (b), applied magnetic field strength (c), and type of metal used to construct the stent (d) on the capture cross section as a function of blood flow velocity are shown.

The preceding analyses show that larger particles, higher magnetic composition, and choice of stent material dramatically affect the capture cross section. Note, the use of iron magnetic phases as opposed to more easily fabricated magnetite (Fe₃O₄) phases increases the capture cross section by almost a factor of two. In contrast, the capture cross section is a weak function of the applied magnetic field strength.

In vivo experiments suggest that magnetic particles may aggregate during flow. Aggregated particles can be more efficiently captured (FIG. 6). In the Femlab simulation, aggregates the size of 20 μm can be captured at 50% efficiency in high flow arteries (u_{bo}=100 cm/s).

### EXAMPLE 4

The combination of a magnetizable stent and a medication-containing magnetic particle provides systems and methods for treating any of a wide variety of diseases, disorders and conditions that would benefit from the controlled release of a medication to a localized area in vivo. In particular, these aspects of the invention provide for the targeted, non-invasive, and potentially repetitive treatment of a stent-surrounding cell, tissue, organ or organ system of an organism, such as a human, as well as for the treatment of stent failure, for example, due to a blood clot and/or cellular overgrowth within the stent lumen.

Coronary atherosclerosis is a life-threatening disease that restricts blood flow through the coronary arteries supplying the heart muscle itself with a blood supply. Occlusion of these arteries results in heart muscle ischemia (myocardial infarction) and is a significant world-wide health concern of humans, with considerable health-related resources devoted to its treatment. Coronary angioplasty is a catheter-based procedure performed by an interventional cardiologist in order to open up a blocked coronary artery and restore blood flow to the heart muscle. Angioplasty is an alternative treatment to coronary artery bypass surgery. Angioplasty is less invasive, less expensive, and faster to perform than bypass surgery, with the patient usually returning home the next day. The main disadvantage of coronary angioplasty is that, approximately 20%-30% of the time, the artery closes up again within six months, a process called restenosis. Treatment of restenosis requires another angioplasty procedure and is more problematic (sometimes requiring open heart surgery), and often less successful, than placement of a first coronary stent. The systems and methods of this aspect of the invention can be used to reduce, or eliminate, these drawbacks to coronary angioplasty, including repeated angioplasty, as a treatment for coronary artery disease.

A magnetizable stent made of stainless steel 405, or a higher grade as would be known in the art, is manufactured with dimensions suitable to fit a human coronary artery, i.e., an inner diameter of 1.5 to 4.0 mm, wall thickness of 1.0 or 1.5 mm; and variable length of 5 to 15 mm. Designs vary among straight, curved, and branched forms in order to conform to vascular anatomy and to enhance the radial strength of the stent while retaining longitudinal flexibility. Deployment of the coronary stent in vivo is based on self- or balloon-mediated expansion in situ, with the stent maintained in a compressed form during placement by coronary angioplasty.

The stent is placed into a coronary artery using a conventional transarterial approach involving a small catheter-like instrument that is advanced intraarterially from the groin artery (transfemoral) to the coronary artery, guided by x-ray monitoring. Once located at the target site, the stent is fixed in position by initially inflating a balloon to open the coronary artery and then by releasing the self-expandable stent.

To treat or prevent the restenosis often found to accompany coronary angioplasty non-invasively, the stent narrowing is treated by administration, e.g., intravenous injection, of a medication-containing magnetic particle, generally as described herein. The magnetic particles exhibit diameters in the range of 100-5,000 nm containing 10-30% magnetite and are coated with a co-polymer block of PLA/PLGA-PEG. Tissue plasminogen activator is encapsulated within the magnetic particles.

For example, acute blood clots (thrombosis) within the stent-containing arterial segment, a common problem encountered with current stent technology, is treated with a bolus injection of medication-containing magnetic particles. A single administration of 100 mg of biodegradable and biocompatible magnetic particles (mixed in 50 ml buffered 0.9% sodium chloride solution) contains sufficient tissue plasminogen activator (tPA; a fibrinolytic agent or “clot buster”) to lyse an acute blood clot within the stent. The bolus injection of 100 mg of particles will not occlude coronary vessels. Maximal loading of the magnetic particles with tPA is expected to result in encapsulation of 1% (w/w) tPA in the particles. Optimal retention of magnetic particles by the magnetizable stent is expected to be at least 50% of the particles, and optimal release of the encapsulated tPA is expected to yield about 50% of the total tPA, with 90% of the released tPA being active. Accordingly, it is expected that the methods and systems of the invention will produce a local concentration of tPA of about up to about 225 μg/ml, which is sufficient to achieve therapeutic efficacy in treating coronary thrombosis in humans.

Shortly prior to bolus injection, a mobile neodymium iron boron magnet is positioned at the anterior (front) chest wall directly above the heart region. After injection; the circulating magnetic particles are trapped and concentrated at the stent site due to the strong focal magnetic field induced by the externally placed parent magnet. A second function, typically supplied by a second device, is the provision of ultrasound energy. A second device capable of emitting ultrasound energy and also positioned at the external chest wall and in proximity to the magnet delivers simultaneously focused ultrasound energy (e.g., 10 MHz; 0.5 to 1 watt) to the target stent site (conveniently under ultrasonic guidance). Interaction of the ultrasound beam with the magnetic particles within the stent region triggers release of encapsulated plasminogen activator concentrated at the stent site, with subsequent lysis of the target clot. Without wishing to be bound by theory, the ultrasound energy is believed to heat the particles, without appreciable heating of the particle environment, sufficiently to result in particle degradation sufficient to release encapsulated compounds and compositions, including proteins. The ultrasound energy for non-cranial applications may be any radiofrequency, but is typically in the range of 2.5-15 MHz. The energy may be delivered on a continuous or pulsed schedule. Advantageously, using ultrasound to release encapsulated active compounds from the magnetic particles facilitates visualization of the target cell, tissue, organ or organ system. Thus, clot lysis is targeted to the area of interest (stent occlusion) but is achieved non-invasively (as the plasminogen activator is injected intravenously, that is, systemically). Moreover, use of ultrasound provides a positive control over the rate of release, unlike the steady leaching of, e.g., hydrophobic therapeutics, from PLGA/PLA-coated spheres that are known in the art.

Those of skill in the art will recognize that magnetizable stents of varying magnetizable compositions, sizes and shapes may be placed in any vessel within an organism such as man, or in non-vessel regions (e.g., lumina) of the body. not only the inner stent lumen can be treated but also the magnetic interactions of external magnet and stent/seed will enable the trapped medicated particles to be pulled into the stent/seed surrounding tissue and hence we have a novel, actively guided tissue delivery system

Moreover, the use of magnetic particles to deliver a medication to the stent target site increases the versatility of the systems and methods in that one of skill will recognize that a great variety of medications are amenable to association with a magnetic particle through covalent attachment to a surface coating of a particle or to the surface of the particle itself, to non-covalent attachment thereto, to incorporation in a particle (e.g., co-polymerization, or co-synthesis in the case of non-polymeric medications, with the particle), or encapsulation within the particle. With the stent/seed in place, repeated dosages are non-invasively delivered using routine administration routes such as injection. In certain embodiments, the multiple dosing is controlled by initially placing a stent/seed pre-loaded with medication-containing magnetic particles, or by delivering a bolus of such particles after stent/seed placement, with localization of the particles through magnetic interaction with the stent. The invention contemplates association of magnetic particles with any part, or all, of the stent/seed, including, but not limited to, the internal luminal surface of a stent, an internal region of a permeable or semi-permeable seed material, and an exterior surface of a stent/seed.

Controlled variation in the magnetic field by manipulation of an external magnetic field source allows partial release of the sequestered particles containing the medication, a process that can be repeated to provide multiple doses in a relatively non-invasive manner.

### EXAMPLE 5

One example of the methods and systems of the invention drawn to the use of medication-containing magnetic particles in conjunction with a magnetizable seed is an irresectable brain tumor expected to be fatal. To treat such an inoperable tumor, a stereotactic procedure similar to the stereotactic biopsy of brain tumor typically used to diagnose such a disease is employed. In particular, a magnetizable seed is stereotactically implanted into the tumor-containing brain tissue in an appropriate surgical procedure. Subsequent to seed placement, sustained, or chronic, drug treatment (e.g., chemotherapy or radiotherapy) is initiated by administration of medication-containing magnetic particles.

The magnetizable implantable seed consists of small stainless steel (405 or higher grades) implantable pellets. Seeds are manufactured with dimensions suitable for stereotactic implantation and tissue deposition. For example, stainless steel pellets having an average diameter of 2 mm are implanted (10 mg) into the affected brain tissue by surgical placement.

Subsequent treatment sessions, i.e., administration of medication-containing magnetic particles, will target the affected tissue by virtue of the magnetic attraction of the particles for the positioned seed. Treatment schedules will vary in individual cases, dependent upon the values for result-affective variables known in the art. Those of skill in the art are able to assess these variables (e.g., patient age, health, weight, prior medical history, other medications being consumed, and the like) and determine a proper treatment schedule using routine procedures.

Each administration of the therapeutic may involve the positioning of a magnet, e.g., a mobile neodymium iron boron magnet, at the skull and above the brain tumor region. Such a magnet will establish a magnetic field within the brain, which is locally enhanced by the implanted seed pellets within the tumor-containing tissue or the tumor tissue itself. Typically following the positioning of the external magnet, 100 mg of magnetic particles (buffered in sodium chloride, i.e., physiological saline), which contain the encapsulated chemotherapeutic medication (e.g., 5 mg methotrexate) is administered. The injected particles, reaching the brain tumor via the systemic blood circulation, are trapped and concentrated within the magnetic field of the implanted seed pellets. Triggered release of the chemotherapeutic agent from the magnetic carrier is obtained with a 2 MHz ultrasound beam through the skull targeted at the tumor and seed site. Typically, ultrasound-induced release of the medication, or other compounds in other embodiments of the invention, is accomplished by ultrasound bombardment at 2 MHz or lower for skull penetration. The ultrasound energy can be delivered on a continuous or pulsed schedule.

These treatment methods involve the initial placement of a magnetizable seed, such as the stainless steel pellets. All procedures subsequent to such placement are non-invasive in nature (e.g., intravenous injection) and may be repeated. In some embodiments, moreover, a single bolus of medication-containing magnetic particles may be administered and sequestered at the site of the seed. Subsequent manipulation of an external magnetic field generator may then be used to achieve partial release of the bound particles, a process that can be repeated to achieve multiple dosing scheduling without even requiring the administration of such particles by injection or the like.

One of skill in the art will readily appreciate that the magnetizable seed may be of any size, shape or number, provided that it retains the properties of being magnetizable and biocompatible. Moreover, one of skill will recognize that such magnetizable seeds, and stents, are also useful with magnetic particles associated with diagnostic agents or compounds that either specifically bind deleterious substances, such as toxins, or inactivate or inhibit such compounds. Those aspects of the invention drawn to the use of a magnetizable seed or stent in conjunction with a functionalized magnetic particle for the effective removal of a deleterious substance may further involve the physical removal of the magnetic particle-deleterious substance complex, optionally facilitated by an external magnetic filtration device.

### REFERENCES

T. M. Allen, et al., “Pharmacokinetics of long-circulating liposomes,” Adv. Drug Deliv. Rev., 16, 267-284, 1995.
S. K. Huang, et al., “Pharmacokinetics and therapeutics of sterically stabilized liposomes in mice bearing C-26 colon carcinoma” Cancer Research, 52, 6774, 1992.
Volkonsky V. A. et al, “Magnetically responsive composition for carrying biological substances and methods of production,” U.S. Pat. Nos. 5,549,915; 5,651,989; 5,705,195; 5,200,547; 1994-1998.
U. Häfeli, et al., “Magnetically directed poly(lactic acid) ⁹⁰Y-microcapsules: novel agents for targeted intracavitary radiotherapy” Journal of Biomedical Materials Research, 28, 901, 1994.
Cortex Biochem, Inc. website, product catalog, 2003.
A. Lubbe et al., “Preclinical experiences with magnetic drug targeting: tolerance and efficacy” Cancer Research, 56, pp. 4694-4701, 1991.
Allen & Moase, “Therapeutic opportunities for targeted liposomal drug delivery” Advanced Drug Delivery Reviews 21 pp. 117-133, 1996.
T. Allen et al., “Liposomes with prolonged circulation times: factors affecting uptake by reticuloendothelial and other tissues” Biochimica et Biophysica Acta, 981 pps 27-35, 1989.
S. Dunn et al., “Polystyrene-poly (ethylene glycol) (PS-PEG2000) particles as model systems for site specific drug delivery. 2. The effect of PEG surface density on the in vitro cell interaction and in vivo biodistribution.” Pharma Research, 11 pps 1016-1022, 1994.
R. Gref et al., “Biodegradable long-circulating polymeric nanospheres” Science 263 pps 1600-1603, 1994.

Each of the references cited in this application is hereby incorporated by reference in its entirety.

While the invention has been described in terms of specific embodiments, it is understood that variations and modifications will occur to those skilled in the art. Accordingly, only those limitations appearing in the appended claims should be placed upon the invention.

We claim:

1. A method for controlling the administration of an effectively magnetized compound selected from the group consisting of a medication, a diagnostic agent, a specific binding partner for a deleterious substance and an inhibitor of a deleterious substance comprising:
(a) introducing a magnetizable device selected from the group consisting of a magnetizable stent and a magnetizable seed into an organism in need, wherein said device is magnetically associated with said compound; and
(b) establishing a magnetic field across said device, thereby capturing said compound.

2. The method according to claim 1 wherein said compound is effectively magnetized by attachment to a biocompatible magnetic particle comprising a mean diameter between 100 and 5,000 nanometers and exhibiting an in vivo half-life of at least fifteen minutes.

3. The method according to claim 2 wherein establishing a magnetic field comprises application of an external magnetic field across said device.

4. The method according to claim 2 wherein said device is introduced into an organism in need by a procedure selected from the group consisting of surgical implantation, catheter-mediated implantation, cannula-mediated implantation and stereotactic placement.

5. The method according to claim 2 wherein said compound is a medication selected from the group consisting of a chemotherapeutic, a radioactive isotope, a fibrinolytic agent and an anti-platelet aggregation drug.

6. The method according to claim 2 wherein said compound is a specific binding partner for a deleterious substance selected from the group consisting of a specific anti-deleterious substance antibody, a fragment of an anti-deleterious substance antibody specifically binding said deleterious substance and a radioisotope chelator.

7. The method according to claim 2 wherein said magnetic particle comprises a magnetizable material selected from the group consisting of magnetite (Fe₃O₄), maghemite (g-Fe₂O₃), metallic iron, cobalt, nickel, permalloy, cobalt ferrite (CoFe₂O₄), NdFeB, SmFe₂, TbFe₂, TbDyFe, NdCo₅, SmCo₅, LaCo₅, CeCo₅ and PrCo₅.

8. The method according to claim 2 wherein said magnetic particle is coated with a biocompatible polymer selected from the group consisting of poly (lactic co-glycolic acid), poly (lactic acid), a linear polyethylene glycol, a branched polyethylene glycol, a propylene glycol, dextran and albumin.

9. A method for controlling the administration of an effectively magnetized compound selected from the group consisting of a medication, a diagnostic agent, a specific binding partner for a deleterious substance and an inhibitor of a deleterious substance comprising:
(a) introducing a magnetizable device selected from the group consisting of a magnetizable stent and a magnetizable seed into an organism in need, wherein said device is associated with said compound;
(b) administering a therapeutically effective amount of an effectively magnetizable compound to said organism; and
(c) establishing a magnetic field across said device, thereby capturing said compound.

10. The method according to claim 9 wherein said compound is effectively magnetized by attachment to a biocompatible magnetic particle comprising a mean diameter between 100 and 5,000 nanometers and exhibiting an in vivo half-life of at least fifteen minutes.

11. The method according to claim 10 wherein said administering is accomplished by a procedure selected from the group consisting of intraarterial injection, intravenous injection, intramuscular injection, intraperitoneal injection, subcutaneous injection, transdermal delivery, inhalation, intraluminal spraying and topical administration.

12. The method according to any one of claims 2 or 10 wherein said compound is selected from the group consisting of a specific binding partner for a deleterious substance and an inhibitor of a deleterious substance, said method further comprising removal of said compound attached to said magnetic particle after a time period sufficient for said compound to bind to said deleterious substance, thereby removing said deleterious substance.

13. The use of a magnetic particle according to any one of claims 2 or 10 attached to a medication in the preparation of a medicament for the treatment of a disease, disorder or condition in an organism in need.

14. The use of a magnetic particle according to any one of claims 2 or 10 attached to a diagnostic agent in the preparation of a medicament for the diagnosis of a disease, disorder or condition in an organism in need.

15. A kit comprising a compound attached to a magnetic particle according to any one of claims 2 or 10 and a set of instructions for administration of said compound to treat or diagnose a disease in an organism in need.

16. A method for removing a deleterious substance from an organism comprising:
(a) administering a biocompatible magnetic particle to an organism under conditions wherein said particle binds to said substance, and wherein said particle has an in vivo half-life of at least thirty minutes; and
(b) removing said particle from said organism by exposing said particle to a magnetic field, thereby removing said deleterious substance.

17. The method according to claim 16 wherein said particle specifically binds said deleterious substance.

18. The method according to claim 17 wherein said particle has a diameter between about 100 and 5,000 nanometers and exhibits an in vivo half life of at least 15 minutes.

19. The method according to claim 18 wherein said deleterious substance is selected from the group consisting of a bacterial cell, a virus, a DNA, a RNA, a prion, a radionuclide, a radioactive material and a metal.

20. The method according to claim 16 wherein said deleterious substance is a toxin.

21. The method according to claim 20 wherein said toxin is selected from the group consisting of Abrin, Adenylate cyclase, Aerolysin, Aflatoxin, Alpha toxin, Adroctonin, Anthrax toxin, Botulinum toxin (A), Botulinum toxin (B), Botulinum toxin (C), Botulinum toxin (D), Botulinum toxin (E), Botulinum toxin (F), Botulinum toxin (G), C2 toxin, C3 toxin, Cholera enterotoxin CLDT, CFN, Conotoxin-alpha, Conotoxin-alpha-A, Conotoxin-psi, Conotoxin-omega, Conotoxin-mu, Conotoxin-delta, Conotoxin-kappa, Cytotoxic necrotizing factor type I, oxynivalenol, Dermonecrotic toxin, Diacetoxyscirpenol, Diptheria toxin, EAST, Epsilon toxin, Equinatoxin II, Erythrogenic toxin, Exfoliatin toxin, Exotoxin A, Flavocetin, Hemolysin, Huwentoxin-I, Huwentoxin-II, Huwentoxin-UV, Iota toxin, Leukocydin F, Listeriorlysin O, LT toxin, Mastoparan, Nivalenol, Nodularin, Perfringolysin O, Perfringens enterotoxin, Pertussis toxin, Pneumolysin, Pyrogenic exotoxin, Ricin, Saxitosin, Scorpion toxin, Shiga, ST toxin, Staphylococcus enterotoxin, Streptolysin O, T-2 toxin, Tetanus toxin, Tetradotoxin, Toxic shock syndrome toxin, Toxin A, Toxin B and a radionuclide.

22. The method according to claim 21 wherein said toxin is selected from the group consisting of Anthrax toxin, Botulinum toxin (A), Botulinum toxin (B), Botulinum toxin (C), Botulinum toxin (D), Botulinum toxin (E), Botulinum toxin (F), Botulinum toxin (G), Ricin, Saxitosin, Staphylococcus enterotoxin, and Tetradotoxin.

23. The method according to claim 20 wherein said toxin is selected from the group consisting of americium-241, plutonium-239, plutonium-240, plutonium-238, uranium-238, uranium-235, europium-154, europium-155, cesium-137, strontium-90, iodine-131, iodine-125, iodine-129, technetium-99, neptunium-237, curium-244, rhenium-188, radium-228, radium-226 and cobalt-60.

24. The method according to claim 16 wherein said particle has a paramagnetic core.

25. The method according to claim 24 wherein said core is encased in a compound selected from the group consisting of polystyrene, poly (lactic acid) and poly (lactic-glycolic acid).

26. The method according to claim 16 wherein said particle is effectively coated with a polyalkylene glycol.

27. The method according to claim 26 wherein said compound is selected from the group consisting of polyethylene glycol and polypropylene glycol.

28. The method according to claim 16 wherein said particle further comprises a specific binding partner for said substance.

29. The method according to claim 28 wherein said binding partner is selected from the group consisting of a receptor specific for a ligand, a ligand specific for a receptor, a ligand specific for a radionuclide, an antigen, a hapten and an antibody.

30. The method according to claim 16 wherein said organism is selected from the group consisting of a multicellular plant, a fish, an amphibian, a reptile and a mammal.

31. The method according to claim 16 wherein said organism is a human.

32. The method according to claim 16 wherein said administering is selected from the group consisting of injection, surgical implantation, catheterization, cannulation, oral delivery, anal delivery and topical delivery.

33. The method according to claim 32 wherein said administering is continuous.

34. The method according to claim 16 wherein removing said particle is achieved by removing said particle from a biological fluid.

35. The method according to claim 34 wherein said biological fluid is selected from the group consisting of blood, plasma and lymph.

36. The method according to claim 35 wherein said biological fluid is blood.

37. The method according to claim 16 wherein said removing is achieved using a magnetic field gradient.

38. The method according to claim 37 wherein said magnetic field is an electromagnetic field.

39. The method according to claim 37 wherein the step of removing comprises:
(a) circulating blood through a closed-loop catheter system in fluid communication with the bloodstream of said organism;
(b) exposing the blood to a pre-defined magnetic field gradient, thereby impeding the flow of said particle in said blood; and
(c) returning the blood to said organism.

40. The method according to claim 16 wherein the substance is an endogenous substance.

41. A method for decreasing the deleterious activity of a substance in an organism by modulating the activity of said substance, comprising administering a biocompatible magnetic particle to an organism under conditions wherein said particle binds to said substance, wherein said particle has an in vivo half-life of at least thirty minutes, and wherein said bound substance exhibits detectably decreased deleterious activity, thereby decreasing the deleterious activity of said substance.

42. A method of diagnosing a deleterious substance-induced condition in an organism comprising:
(a) administering a biocompatible magnetic particle to an organism under conditions wherein said particle binds to said substance, and wherein said particle has an in vivo half-life of at least fifteen minutes;
(b) removing said particle from said organism by exposing said particle to a magnetic field; and
(c) identifying said deleterious substance, thereby diagnosing said condition.

43. A kit comprising a compound attached to a magnetic particle according to any one of claims 16, 41 or 42 and a set of instructions for administration of said compound to treat or diagnose a disease in an organism in need.
